(19) United States
(12) Patent Application Publication
(10) Pub. No.: US 2012/0095185 A1
(43) Pub. Date: Apr. 19, 2012

(54) PROCESS FOR CHELATING COPPER IONS USING CB-TE2A BIFUNCTIONAL CHELATE

(75) Inventors: Garry E. KIEFER, Richardson, TX (US); Cara L. Ferreira, Surrey (CA); Paul Jurek, Red Oak, TX (US)

(73) Assignee: NORDION (CANADA) INC., Kanata (CA)

(21) Appl. No.: 13/237,507

(22) Filed: Sep. 20, 2011

Related U.S. Application Data
(60) Provisional application No. 61/384,608, filed on Sep. 20, 2010.

(51) Int. Cl. C07D 487/06; C07K 14/00; C07K 2/00; C07K 16/00; C07F 1/08; C07K 14/475; B82Y 5/00

(52) U.S. Cl. 530/300; 540/472; 534/10; 540/465; 530/400; 530/399; 530/391.3; 977/773; 977/906

(57) ABSTRACT

An isolated conformational isomer of a bifunctional chelating agent of the formula (I):

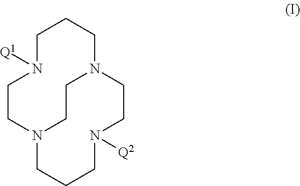

wherein the variables Q¹ and Q² are as defined in the description of the present application. Also described is a complex of the above chelating agent to an ion of a stable or radioactive metal; a conjugate of the complex covalently attached to a biological carrier; and a pharmaceutical composition containing the conjugate.

### FIELD OF INVENTION

The present invention relates to conformational isomers of a bifunctional chelating agent, complexes of these chelating agents with metal ions, and conjugates of these complexes with a biological carrier. More particularly, the present invention relates to a conformational isomer of CB-TE2A for chelating radiometals useful in molecular imaging and therapy, in particular, radioisotopes of copper such as ⁶⁴Cu or ⁶⁷Cu.

### BACKGROUND OF THE INVENTION

Copper has several radioisotopes of interest in the development of radiopharmaceuticals such as Cu-61, Cu-62, Cu-64 and Cu-67.[1] Cu-61 and Cu-62 are positron emitting isotopes with half-lives of about 3.35 h and 9.5 min, respectively, which can be used for nuclear imaging using positron-emission tomography (PET). Cu-67 is a beta-particle-emitting isotope applicable to radiotherapy. Cu-64 has favorable emission characteristics (t_{1/2}=12.7 h, 17.4% β⁺, Eₘₐₓ0.656 MeV; 39% β⁻, Eₘₐₓ=0.573 MeV) for potential use in both nuclear imaging and radiotherapy. The relatively short positron range of Cu-64 is ideal for use in high resolution positron-emission tomography (PET) [1], while the beta-particle emission is applicable to internal source radiotherapy, such as radioimmunotherapy. The longer half-life of Cu-64 compared to other positron-emitting radioisotopes is advantageous for developing PET imaging agents with larger biomolecules, such as proteins and monoclonal antibodies.[2, 3] The potential to use the same element for both imaging and therapy eliminates concerns raised by other imaging—therapy complements, such as In-111 and Y-90, where structural differences may influence the biological equivalence of the two agents.[4-6]

In order to attach a metal radioisotope to a biologically recognized targeting molecule, coordinating groups, e.g., a bifunctional chelate (BFC) must be used. The BFC is used to chelate the radioisotope and form a stable complex, protecting the metal in vivo from transchelation to proteins and other endogenous ligands. Either before or after the chelation of the radioisotope, the BFC can be covalently attached to the targeting biological molecule of choice, such as a peptide or an antibody.

Several different BFCs have been examined for use with Cu radioisotopes. Macrocyclic chelates are preferred, as acyclic chelates have been shown to lack kinetic stability. [7-9] 1,4,7,10-Tetraazacyclododecane-1,4,7,10-tetraacetic acid (DOTA) has been the most widely used macrocyclic chelate in radiopharmaceutical research and development with metal radioisotopes, including Cu radioisotopes,[10] but Cu-64 radiolabeling of DOTA has slow reaction kinetics and forms a complex with only moderate stability in vivo.[11, 12] Other BFC's have been shown to have either better radiolabeling kinetics or higher stability, but only a few BFCs have been reported to have both these qualities; 1-N-(4-aminobenzyl)-3,6,10,13,16,19-hexaazabicyclo(6.6.6)eicosane-1,8-diamine (SarAr),[13] 1-Oxa-4,7,10-triaazacyclododecane-5-S-(4-isothiocyanatobenzyl)-4,7,10-triacetic acid (Oxo-DO3A) and 3,6,9,15-tetraazabicyclo[9.3.1]pentadeca-1(15),11,13-triene-4-S-(4-isothiocyanatobenzyl)-3,6,9-triacetic acid (PCTA).[14]

The syntheses of cross-bridged 1,4,8,11-tetraazacyclotetradecane-1,4,8,11-tetraacetic acid (CB-TE2A) and derivatives have been previously disclosed.[15-17] The synthesis of the bifunctional derivative of the formula:

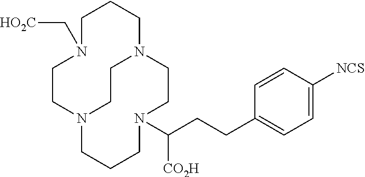

was disclosed in U.S. Patent Application Publication No. 2006/0062728 A1, entitled “Multifunctional cross-bridged tetraza macrocyclic compounds and methods of making and using” but no details on the Cu-64 radiolabeling properties or examples of Cu-64 radiolabeling were provided. Both CB-TE2A and derivatives thereof have been applied to Cu-64 radiolabeling because they have been shown to have superior stability compared to other chelates that are applicable to Cu radioisotope chelation. [11, 18, 19] Reported shortcomings of CB-TE2A for Cu-64 radiolabeling is that it requires harsher radiolabeling conditions than other chelates, such as high pH (up to pH 8) and heating at up to 95° C.[20-22] These conditions have greatly limited the utility of CB-TE2A and CB-TE2A derivatives with respect to radiolabeling sensitive biomolecules such as antibodies and many proteins. Therefore, there is a need for a CB-TE2A bifunctional derivative that can be efficiently radiolabeled under mild room temperature conditions.

### SUMMARY OF THE INVENTION

The present invention relates to conformational isomers of a bifunctional chelating agent, complexes of these chelating agents with metal ions, and conjugates of these complexes with a biological carrier. More particularly, the present invention relates to a conformational isomer of CB-TE2A for chelating radiometals useful in molecular imaging and therapy, in particular, radioisotopes of copper such as ⁶⁴Cu or ⁶⁷Cu.

In a first aspect, the present invention provides an isolated conformational isomer of a bifunctional chelating agent of formula (I):

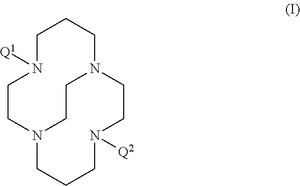

wherein:
Q¹ is —(CHR²)ₚCO₂R³ or —(CHR²)ₚPO₃R⁴R⁵;
Q² is

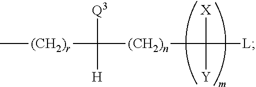

Q³ is —(CHR²)_{w}CO₂R³ or —(CHR²)_{w}PO₃R⁴R⁵;
each R² is independently hydrogen; C₁-C₄ alkyl or (C₁-C₂alkyl)phenyl;
each R³ is independently H, benzyl, C₁-C₄ alkyl, or a protecting group;
R⁴ and R⁵ are independently H, C₁-C₆ alkyl, (C₁-C₂ alkyl)phenyl or a protecting group;
X and Y are each independently hydrogen or may be taken with an adjacent X and Y to form an additional carbon-carbon bond;
m is an integer from 0 to 5 inclusive;
n is an integer from 1 to 5 inclusive;
p is 1 or 2;
r is 0 or 1;
w is 0 or 1;
L is a linker/spacer group covalently bonded to a carbon atom and replaces one hydrogen atom of said carbon atom, said linker/spacer group being represented by the formula:

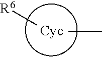

wherein:
   R⁶ is H or an electrophilic, nucleophilic or electron-rich moiety which allows for covalent attachment to a biological carrier, or a synthetic linker which can be attached to a biological carrier, a protected form thereof or a precursor thereof; and
   Cyc represents a linear, branched or cyclic aliphatic moiety, aromatic moiety, aliphatic heterocyclic moiety, or aromatic heterocyclic moiety, each of said moieties optionally substituted with one or more groups which do not interfere with binding to a biological carrier;
or a pharmaceutically acceptable salt thereof,
   wherein the conformational isomer is prepared by a process comprising:

a) reacting a compound of formula:

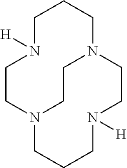

with a compound of formula Q²-LG, wherein Q² is as defined above and LG is a leaving group, to form a mixture of conformational isomers of a mono-alkylated derivative of formula:

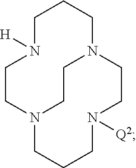

b) reacting the mixture of conformational isomers of the monoalkylated derivative with a compound of formula Q¹-LG, wherein Q¹ is as defined above and LG is a leaving group, to form a mixture of conformational isomers of the compound of formula (I), the mixture of conformational isomers comprising a first conformational isomer and a second conformational isomer; and

c) isolating the first conformational isomer, such as the most polar or the least polar of the mixture of conformational isomers of the compound of formula (I), or

d) forming a solution of the mixture of conformational isomers in a polar solvent, a non-polar solvent, such as chloroform, or a mixture of polar and non-polar solvents that produce a homogenous solution of the conformational isomers, allowing the second conformational isomer to convert to the first conformational isomer and isolating the total amount of the first conformational isomer, and optionally e) hydrolyzing the isolated conformational isomer under basic conditions,

wherein when R⁶ comprises a precursor of an electrophilic group, the process further optionally comprises a step of converting the precursor of the electrophilic group to the electrophilic group,

wherein when R⁶ comprises a precursor or protected form of a nucleophilic group, the process further optionally comprises a step of converting the precursor or protected form of the nucleophilic group to the nucleophilic group,

wherein when R⁶ comprises an electrophilic group, the process further optionally comprises a step of converting the electrophilic group to a nucleophilic group, and

wherein when R⁶ comprises a nucleophilic group, the process further optionally comprises a step of converting the nucleophilic group to an electrophilic group.

In a second aspect, the present invention provides a method of isolating a conformational isomer of a bifunctional chelating agent of formula (I):

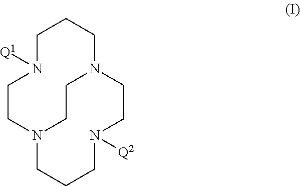

wherein:
Q¹ is —(CHR²)ₚCO₂R³ or —(CHR²)ₚPO₃R⁴R⁵;
Q₂ is

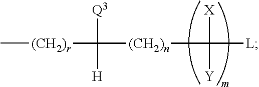

Q³ is —(CHR²)_{w}CO₂R³ or —(CHR²)_{w}PO₃R⁴R⁵;
each R² is independently hydrogen; C₁-C₄ alkyl or (C₁-C₂alkyl)phenyl;
each R³ is independently H, benzyl, C₁-C₄ alkyl, or a protecting group;
R⁴ and R⁵ are independently H, C₁-C₆ alkyl, (C₁-C₂ alkyl)phenyl or a protecting group;
X and Y are each independently hydrogen or may be taken with an adjacent X and Y to form an additional carbon-carbon bond;
m is an integer from 0 to 5 inclusive;
n is an integer from 1 to 5 inclusive;
p is 1 or 2;
r is 0 or 1;
w is 0 or 1;
L is a linker/spacer group covalently bonded to a carbon atom and replaces one hydrogen atom of said carbon atom, said linker/spacer group being represented by the formula:

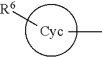

wherein:
   R⁶ is H or an electrophilic, nucleophilic or electron-rich moiety which allows for covalent attachment to a biological carrier, or synthetic linker which can be attached to a biological carrier, a protected form thereof or a precursor thereof; and
   Cyc represents a linear, branched or cyclic aliphatic moiety, aromatic moiety, aliphatic heterocyclic moiety, or aromatic heterocyclic moiety, each of said moieties optionally substituted with one or more groups which do not interfere with binding to a biological carrier;

or a pharmaceutically acceptable salt thereof, the method comprising:

a) reacting a compound of formula:

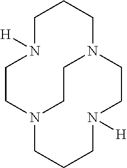

with a compound of formula Q²-LG, wherein is as defined above and LG is a leaving group, to form a mixture of conformational isomers of a mono-alkylated derivative of formula:

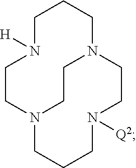

b) reacting the mixture of conformational isomers of the monoalkylated derivative with a compound of formula Q¹-LG, wherein Q¹ is as defined above and LG is a leaving group, to form a mixture of conformational isomers of the compound of formula (I), the mixture of conformational isomers comprising a first conformational isomer and a second conformational isomer, and

c) isolating the first conformational isomer, such as the most polar or the least polar of the mixture of conformational isomers of the compound of formula (I), or

d) forming a solution of the mixture of conformational isomers in a polar solvent, a non-polar solvent, such as chloroform, or a mixture of polar and non-polar solvents that produce a homogenous solution of the conformational isomers, allowing the second conformational isomer to convert to the first conformational isomer and isolating the total amount of the first conformational isomer, and

optionally e) hydrolyzing the isolated conformational isomer under basic conditions,

wherein when R⁶ comprises a precursor of an electrophilic group, the process further optionally comprises a step of converting the precursor of the electrophilic group to the electrophilic group,

wherein when R⁶ comprises a precursor or protected form of a nucleophilic group, the process further optionally comprises a step of converting the precursor or protected form of the nucleophilic group to the nucleophilic group,

wherein when R⁶ comprises an electrophilic group, the process further optionally comprises a step of converting the electrophilic group to a nucleophilic group, and

wherein when R⁶ comprises a nucleophilic group, the process further optionally comprises a step of converting the nucleophilic group to an electrophilic group.

In a third aspect, the present invention provides a method of converting a first conformational isomer of a bifunctional chelating agent of formula (I) into a second conformational isomer of formula (I):

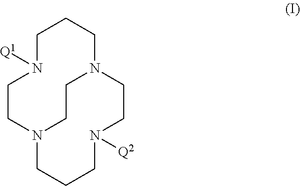

wherein:
Q¹ is —(CHR²)ₚCO₂R³ or —(CHR²)ₚPO₃R⁴R⁵;
Q² is

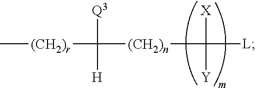

Q³ is —(CHR²)_{w}CO₂R³ or —(CHR²)_{w}PO₃R⁴R⁵;
each R² is independently hydrogen; C₁-C₄ alkyl or (C₁-C₂alkyl)phenyl;
each R³ is independently H, benzyl, C₁-C₄ alkyl, or a protecting group;
R⁴ and R⁵ are independently H, C₁-C₆ alkyl, (C₁-C₂ alkyl)phenyl or a protecting group;
X and Y are each independently hydrogen or may be taken with an adjacent X and Y to form an additional carbon-carbon bond;
m is an integer from 0 to 5 inclusive;
n is an integer from 1 to 5 inclusive;
p is 1 or 2;
r is 0 or 1;
w is 0 or 1;

L is a linker/spacer group covalently bonded to a carbon atom and replaces one hydrogen atom of said carbon atom, said linker/spacer group being represented by the formula:

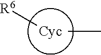

wherein:
   R⁶ is H or an electrophilic, nucleophilic or electron-rich moiety which allows for covalent attachment to a biological carrier, or synthetic linker which can be attached to a biological carrier, a protected form thereof or a precursor thereof; and
   Cyc represents a linear, branched or cyclic aliphatic moiety, aromatic moiety, aliphatic heterocyclic moiety, or aromatic heterocyclic moiety, each of said moieties optionally substituted with one or more groups which do not interfere with binding to a biological carrier;

or a pharmaceutically acceptable salt thereof, the method comprising:

forming a solution of the first conformational isomer and the second conformational isomer in a polar solvent, a non-polar solvent, such as chloroform, or a mixture of polar and non-polar solvents that produce a homogenous solution of the conformational isomers;

allowing the second conformational isomer to convert to the first conformational isomer,

isolating the total amount of the first conformational isomer, and

optionally e) hydrolyzing the isolated conformational isomer under basic conditions,

wherein when R⁶ comprises a precursor of an electrophilic group, the process further optionally comprises a step of converting the precursor of the electrophilic group to the electrophilic group,

wherein when R⁶ comprises a precursor or protected form of a nucleophilic group, the process further optionally comprises a step of converting the precursor or protected form of the nucleophilic group to the nucleophilic group,

wherein when R⁶ comprises an electrophilic group, the process further optionally comprises a step of converting the electrophilic group to a nucleophilic group, and

wherein when R⁶ comprises a nucleophilic group, the process further optionally comprises a step of converting the nucleophilic group to an electrophilic group.

The present invention also relates to the method of the third aspect of the present invention, wherein the first conformation isomer is relatively more polar than the second conformational isomer.

The present invention also relates to the above-defined isolated conformational isomer and the methods of the second and third aspects of the present invention, wherein Q² is

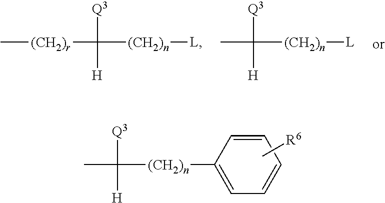

and n, r, L, Q³ and R⁶ are as defined above.

The present invention also relates to the isolated conformational isomer and the methods of the second and third aspects of the present invention described above, wherein Q³ is —(CHR²)_{w}CO₂R³, —CH₂CO₂R³ or —CO₂R³ and w, R² and R³ are as defined above.

The present invention further relates to the isolated conformational isomer and the methods of the second and third aspects of the present invention defined above, wherein Q¹ is —(CHR²)ₚCO₂R³, —CHR²CO₂R³ or —CH₂CO₂R³ and p, R² and R³ are as defined above.

In particular examples, the isolated conformational isomer is of the formula:

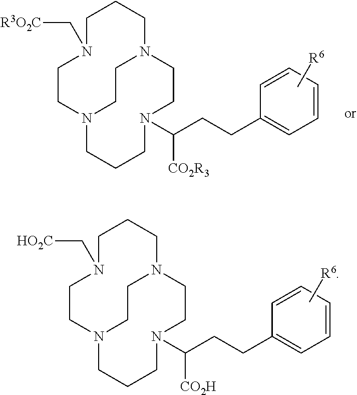

In other examples, R⁶ is NO₂, NH₂, isothiocyanato, semicarbazido, thiosemicarbazido, maleimido, bromoacetamido or carboxylic acid.

In a fourth aspect, the present invention provides a complex comprising the bifunctional chelating agent defined above or a pharmaceutically acceptable salt thereof, and an ion of a stable or radioactive form of Cu.

The present invention also relates to the above-defined complex, wherein the ion is selected from a group consisting of ⁶⁰Cu²⁺, ⁶²Cu²⁺, ⁶⁴Cu²⁺ and ⁶⁷ Cu²⁺.

In a fifth aspect, the present invention provides a conjugate comprising one of the complexes defined above covalently attached to a biological carrier, such as a protein, antibody, antibody fragment, hormone, peptide, growth factor, antigen or hapten.

In a sixth aspect, the present invention provides a process for chelating the isolated conformational isomer defined above with an ion of a stable or radioactive form of Cu, comprising contacting the isolated conformational isomer with the ion and allowing a complex between the isolated conformational isomer and the ion to form.

In a seventh aspect, the present invention provides a pharmaceutical composition comprising the conjugate defined above, and a pharmaceutically acceptable carrier.

In an eighth aspect, the present invention provides a method of therapeutic treatment of a mammal having cancer which comprises administering to said mammal a therapeutically effective amount of the pharmaceutical composition defined above.

The isolated conformational isomers of the present invention are advantageous in that they can be efficiently radiolabeled with a Cu radioisotope in a buffered aqueous solution at room temperature in less than one hour. The conditions required for radiolabeling the isolated conformational isomers of the present invention are a substantial improvement over the reported conditions for radiolabeling of CB-TE2A and derivatives thereof with Cu ions, which involve basic values of pH and a temperature of 95° C. [23-25], and may have a significant impact in the development of radiopharmaceutical agents based on complexes of CB-TE2A derivatives and Cu radioisotopes for nuclear imaging and radiotherapy.

### BRIEF DESCRIPTION OF THE DRAWINGS

FIG. 1A illustrates the ¹H-NMR spectrum corresponding to compound 10.

FIG. 1B illustrates the ¹³C-NMR spectrum corresponding to compound 10.

FIG. 2A illustrates the ESI mass spectrum of a mixture of conformational isomers 12a and 12b.

FIG. 2B illustrates the reverse phase HPLC chromatogram of a mixture of conformational isomers 12a and 12b.

FIG. 2C illustrates the reverse phase HPLC chromatogram of conformational isomer 12b.

FIG. 2D illustrates the reverse phase HPLC chromatogram of conformational isomer 12a.

FIG. 2E illustrates the ESI mass spectrum of conformational isomer 12b.

FIG. 2F illustrates the ESI mass spectrum of conformational isomer 12a.

FIG. 3A illustrates the ¹³C-NMR spectrum of conformational isomer 12b.

FIG. 3B illustrates the ¹³C-NMR spectrum of conformational isomer 12a.

FIG. 4A illustrates the ¹H-NMR spectrum of conformational isomer 13b.

FIG. 4B illustrates the ¹³C-NMR spectrum of conformational isomer 13b.

FIG. 5A illustrates the ¹H-NMR spectrum of conformational isomer 13a.

FIG. 5B illustrates the ¹³C-NMR spectrum of conformational isomer 13a.

FIG. 6 shows a comparison of the aliphatic section of the ¹³C-NMR spectra of conformational isomers 13a and 13b.

FIG. 7A illustrates the aliphatic region of the ¹³C-NMR spectrum of the F1 isomer (12b) in CDCl₃ (bottom) and the aliphatic region of the ¹³C-NMR spectrum of the F2 isomer (12a) in CDCl₃ (top). FIG. 7B illustrates the aliphatic region of the ¹³C-NMR spectra of the F1 isomer (12b) in CDCl₃ at 0 days, 2 days, 6 days, 9 days and 14 days, showing that the F1 isomer (12b) slowly converts to the F2 isomer (12a).

FIG. 8 illustrates reverse phase HPLC chromatograms of conformational isomers 13a and 13b and a mixture of 13a and 13b.

FIG. 9 illustrates the reverse phase HPLC chromatograms relating to Cu-64 radiolabeled 13a and 13b.

FIG. 10 illustrates the uptake of Cu-64 radiolabeled trastuzumab conjugated CB-TE2A in HER2+ (LCC6^{HER-2}) and HER2− (LCC6^{vector}) tumour xenografts determined by PET imaging analysis and biodistribution. In all cases the HER2+ uptake is significantly higher than the HER2− uptake with p values <0.05.

### DETAILED DESCRIPTION OF THE INVENTION

The present invention relates to conformational isomers of a bifunctional chelating agent, complexes of these chelating agents with metal ions, and conjugates of these complexes with a biological carrier. More particularly, the present invention relates to a conformational isomer of CB-TE2A for chelating radiometals useful in molecular imaging and therapy, in particular, radioisotopes of copper such as ⁶⁴Cu or ⁶⁷Cu.

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

As used herein, “complex” refers to a complex of the compound of the invention, e.g. Formula (I), with a metal ion, where at least one metal atom is chelated or sequestered.

The complexes of the present invention can be prepared by methods well known in the art. Thus, for example, see Chelating Agents and Metal Chelates, Dwyer & Mellor, Academic Press (1964), Chapter 7. See also methods for making amino acids in Synthetic Production and Utilization of Amino Acids, (edited by Kameko, et al.) John Wiley & Sons (1974).

The complexes of the present invention can be formed and administered at a ligand-to-metal molar ratio of at least about 1:1, from about 1:1 to about 3:1, or more particularly from about 1:1 to about 1.5:1.

A “conjugate” refers to a metal-ion chelate that is covalently attached to a biological carrier.

As used herein, the term “biological carrier” refers to any biological targeting vector, such as a protein, peptide, peptidomimetic, an antibody, an antibody fragment, a hormone, an aptamer, an affibody molecule, a morpholino compound, a growth factor, an antigen, a hapten or any other carrier, which functions in this invention to recognize a specific biological target site. Antibody and antibody fragment refers to any polyclonal, monoclonal, chimeric, human, mammalian, single chains, dimeric and tetrameric antibody or antibody fragment. Such biological carrier, when attached to a functionalized complex, serves to carry the attached ion to specific targeted tissues.

The term “bifunctional chelating agent” refers to compounds that have a chelant moiety capable of chelating a metal ion and a moiety covalently bonded to the chelant moiety that is capable of serving as a means to covalently attach to a biological carrier for example, a molecule having specificity for tumour cell epitopes or antigens, such as an antibody or antibody fragment. Such compounds are of great utility for therapeutic and diagnostic applications when they are, for example, complexed with radioactive metal ions and covalently attached to a specific antibody.

These types of complexes have been used to carry radioactive metals to tumour cells which are targeted by the specificity of the attached antibody [see, for example, Meares et al., Anal. Biochem. 142, 68-74 (1984); Krejcarek et al., Biochem. Biophys. Res. Commun. 77, 581-585 (1977)].

The bifunctional chelating agents described herein (represented by Formula I) can be used to chelate or sequester a metal ion to form metal-ion chelates (also referred to herein as “complexes”, as defined above). The complexes, because of the presence of the functionalizing moiety (represented by R⁶ in Formula I), or the chelating agents can be covalently attached to a biologically active material, such as dextran, molecules that have specific affinity for a receptor, affibody molecules, morpholino compounds or covalently attached to antibodies or antibody fragments. The term “antibody” refers to a polyclonal antibody, a monoclonal antibody, a chimeric antibody, a heteroantibody, or a fragment thereof. Antibodies used in the present invention may be directed against, for example, cancer, tumours, bacteria, fungi, leukemias, lymphomas, autoimune disorders involving cells of the immune system, normal cells that need to be ablated such as bone marrow and prostate tissue, virus infected cells including HIV, parasites, mycoplasma, differentiation and other cell membrane antigens, pathogen surface antigens, toxins, enzymes, allergens, drugs and any biologically active molecules. Some examples of antibodies are HuM195 (anti-CD33), CC-11, CC-46, CC-49, CC-49 F(ab′)₂, CC-83, CC-83 F(ab′)₂, and B72.3, 1116-NS-19-9 (anti-colorectal carcinoma), 1116-NS-3d (anti-CEA), 703D4 (anti-human lung cancer), 704A1 (anti-human lung cancer) and B72.3. The hybridoma cell lines 1116-NS-19-9, 1116-NS-3d,703D4, 704A1, CC49, CC83 and B72.3 are deposited with the American Type Culture Collection, having the accession numbers ATCC HB 8059, ATCC CRL 8019, ATCC HB 8301, ATCC HB 8302, ATCC HB 9459, ATCC HB 9453 and ATCC HB 8108, respectively.

Antibody fragment includes Fab fragments and F(ab¹)₂ fragments, and any portion of an antibody having specificity toward a desired epitope or epitopes.

Alternatively, the chelators or complexes of the present invention and a biologically active material can both be conjugated to a nanoparticle, or the conjugates of the present invention can be further conjugated to a nanoparticle. The use of nanoparticles as a matrix to which the chelators and complexes of the present invention and a biologically active material can be conjugated is described in S. McNeil, WIREs Nanomed. & Nanobiotechnol. 2009, 1, 264, and U.S. Patent Application Publication No. 2010/0179303, the disclosures of which are incorporated by reference herein.

Complexes of the present invention, which include a radioisotopic metal ion having a relatively short half-life, such as Cu-60, can be conjugated with biological carriers having relatively short or relatively long biological clearance times from a subject. For radioimaging, however, such complexes are typically conjugated to biological carriers having a biological clearance time that is within the lifetime of the short-lived radioisotope so that the systemic background signal produced by unbound conjugated complex can be sufficiently reduced in time to permit imaging of the conjugated complex bound to the target site of the biological carrier. Examples of biological carriers that can be conjugated to complexes of the present invention include peptides or molecular constructs, such as mini-bodies, nano-bodies or affi-bodies. Specific examples of peptides having relatively short clearance times are described in Maecke H R and Reubi J C 2008 Peptide based probes for cancer imaging. J. Nucl. Med. 49:1735-38; Krenning, E P, de Jong M, Kooij P P, Breeman, W A, Bakker W H et. al. 1999 Radiolabelled somatostatin analogue(s) for peptide receptor scintigraphy and radionuclide therapy. Ann. Oncol. 10 Suppl 2:S23-29; Haubner R and Decristoforo C. 2009 Radiolabelled RGD peptides and peptidomimetics for tumour targeting. Front. Biosci. 14:872-86; Ananias H J, de Jong M, Dierckx R A, et al. 2008 Nuclear imaging of prostate cancer with gastrin-releasing-peptide-receptor targeted radiopharmaceuticals. Curr. Pharm. Des. 14(28) 3033-47; Breeman W A, Kwekkeboom D J, de Blois E, de Jong M, et al. 2007 Radiolabelled regulatory peptides for imaging and therapy. Anticancer Agents Med. Chem. 7(3):345-57; Schroeder R P, van Weerden W M, Bangma C, et al. 2009 Peptide receptor imaging of prostate cancer with radiolabelled bombesin analogues. Methods 48(2):200-4, the disclosures of which are incorporated by reference herein.

When using the term “radioactive metal chelate/antibody conjugate” or “conjugate”, the “antibody” is meant to include whole antibodies and/or antibody fragments, including semisynthetic or genetically engineered variants thereof. Such antibodies normally have a highly specific reactivity.

The antibodies or antibody fragments which may be used in the conjugates described herein can be prepared by techniques well known in the art. Highly specific monoclonal antibodies can be produced by hybridization techniques well known in the art, see for example, Kohler and Milstein [Nature, 256, 495-497 (1975); and Eur. J. Immunol., 6, 511-519 (1976)]. Such antibodies normally have a highly specific reactivity in the antibody targeted conjugates, antibodies directed against any desired antigen or hapten may be used. Preferably the antibodies which are used in the conjugates are monoclonal antibodies, or fragments thereof having high specificity for a desired epitope(s).

As used herein, “pharmaceutically-acceptable salt” means any salt or mixture of salts of a complex or conjugate of formula (I) which is sufficiently non-toxic to be useful in therapy or diagnosis of animals, preferably mammals. Thus, the salts are useful in accordance with this invention. Representative of those salts formed by standard reactions from both organic and inorganic sources include, for example, sulfuric, hydrochloric, phosphoric, acetic, succinic, citric, lactic, maleic, fumaric, palmitic, cholic, palmoic, mucic, glutamic, gluconic, d-camphoric, glutaric, glycolic, phthalic, tartaric, formic, lauric, steric, salicylic, methanesulfonic, benzenesulfonic, sorbic, picric, benzoic, cinnamic acids and other suitable acids. Also included are salts formed by standard reactions from both organic and inorganic sources such as ammonium or 1-deoxy-1-(methylamino)-D-glucitol, alkali metal ions, alkaline earth metal ions, and other similar ions. Particularly preferred are the salts of the complexes or conjugates of formula (I) where the salt is potassium, sodium or ammonium. Also included are mixtures of the above salts.

The present invention may be used with a physiologically acceptable carrier, excipient or vehicle therefor. The methods for preparing such formulations are well known. The formulations may be in the form of a suspension, injectable solution or other suitable formulations. Physiologically acceptable suspending media, with or without adjuvants, may be used.

An “effective amount” of the formulation is used for diagnosis or for therapeutic treatments of diseases. The dose will vary depending on the disease and physical parameters of the animal, such as weight. In vivo diagnostics are also contemplated using formulations of this invention.

The chelates of the present invention are useful for binding radioisotopes to biological targeting molecules in order to produce constructs for molecular imaging and therapy, more specifically to produce constructs comprising copper radioisotopes for molecular imaging.

Other uses of some of the chelates of the present invention may include the removal of undesirable metals (i.e. iron) from the body, attachment to polymeric supports for various purposes, e.g. as diagnostic agents, and removal of metal ion by selective extraction.

The free acid of the compounds of formula (I) may be used, also the protonated form of the compounds, for example when the carboxylate is protonated and/or the nitrogen atoms, i.e., when the HCl salt is formed.

The complexes so formed can be attached (covalently bonded) to an antibody or fragment thereof and used for therapeutic and/or diagnostic purposes. The complexes and/or conjugates can be formulated for in vivo or in vitro uses. A particular use of the formulated conjugates is the diagnosis of diseased states (e.g., cancer) in animals, especially humans.

Biotargeted radiopharmaceuticals that employ the chelating agent (ligand) of the present invention to secure a metal radionuclide can be prepared by two methods: 1) Pre-complexation—the metal-ligand complex (chelate) can first be prepared followed by covalent attachment of the chelate to a biotargeting group, for example a monoclonal antibody; 2) Post-complexation—a covalent conjugate between the ligand and the biotargeting molecule can be prepared in a first step followed by introduction and complexation of the metal radionuclide. Both methods have merits and shortcomings. Method 1 is appealing from the standpoint that forcing conditions can be utilized to facilitate complexation however subsequent attachment of the complex to a targeting vector requires more elaborate chemical transformation that can be difficult to perform rapidly in a hospital setting. In contrast, method 2 is desirable since it allows the more intricate chemistry required for conjugation of the ligand and targeting vector to be performed in a controlled environment without time constraints introduced by the radionuclide. The complexation step can then be conducted onsite at the hospital pharmacy by clinical technicians; however, this step can be problematic since the ligand-bound conjugate is much more sensitive to rigorous conditions that favor rapid and complete complexation.

Of the two approaches for preparing biotargeted radiopharmaceuticals, the post-complexation strategy is clearly the most desirable if appropriate ligands and/or conditions can be devised that facilitate rapid and complete incorporation of the radionuclide. In addition, structural and conformational components can be introduced that can minimize kinetic barriers to complexation. For example, molecular architecture which can enhance pre-organization of the ligand binding site toward the necessary conformational requirements of the metal ion should produce faster complexation kinetics.

The bifunctional chelating agents described herein (represented by formula I) are designed to form thermodynamically stable and kinetically inert complexes with the transition group series of metals. Complexation kinetics can be modulated by altering backbone structural rigidity, electronic character of the coordinate donor atoms, and conformational accessibility of the metal-binding site.

While not wishing to be bound by theory, it is believed that kinetic advantages associated with the present invention are a function of structural modifications that lead to preferred molecular geometries (pre-organization) which match ligating requirements of the metal. In this manner the ligand-metal binding event is accelerated without the need for harsh reaction conditions.

In the context of bifunctional chelating agents, the generation of optimal pre-organized ligand structures conducive to rapid complexation kinetics is significantly influenced by the judicious placement of the linking group. In this manner, the linking group can be engineered to assume a position distant from the metal-binding site during the initial stages of the metal-docking process followed by the adoption of a secondary conformation induced by complexation that effectively shields the metal from reversible dissociation pathways. The positional orientation of the linking group also affects the electronic nature of the coordinate donor atoms and their juxtaposed lone pair electrons which are critical for satisfying the geometric requirements of the metal ion.

The present invention also includes formulations comprising the conjugates of this invention and a pharmaceutically acceptable carrier, especially formulations where the pharmaceutically acceptable carrier is a liquid.

The present invention is also directed to a method of therapeutic treatment of a mammal having cancer which comprises administering to said mammal a therapeutically effective amount of the formulation of this invention.

Thus, the present invention may be practiced with the conjugate of the present invention being provided in a pharmaceutical formulation, both for veterinary and for human medical use. Such pharmaceutical formulations comprise the active agent (the conjugate) together with a physiologically acceptable carrier, excipient or vehicle therefor. The methods for preparing such formulations are well known. The carrier(s) must be physiologically acceptable in the sense of being compatible with the other ingredient(s) in the formulation and not unsuitably deleterious to the recipient thereof. The conjugate is provided in a therapeutically effective amount, as described above, and in a quantity appropriate to achieve the desired dose.

This invention is used with a physiologically acceptable carrier, excipient or vehicle therefor. The formulations may be in the form of a suspension, injectable solution or other suitable formulations. Physiologically acceptable suspending media, with or without adjuvants, may be used.

The formulations include those suitable for parenteral (including subcutaneous, intramuscular, intraperitoneal, and intravenous), oral, rectal, topical, nasal, or ophthalmic administration. Formulations may be prepared by any methods well known in the art of pharmacy. Such methods include the step of bringing the conjugate into association with a carrier, excipient or vehicle therefor. In general, the formulation may be prepared by uniformly and intimately bringing the conjugate into association with a liquid carrier, a finely divided solid carrier, or both, and then, if necessary, shaping the product into desired formulation. In addition, the formulations of this invention may further include one or more accessory ingredient(s) selected from diluents, buffers, binders, disintegrants, surface active agents, thickeners, lubricants, preservatives, and the like. In addition, a treatment regime might include pretreatment with non-radioactive carrier.

Injectable compositions of the present invention may be either in suspension or solution form. In the preparation of suitable formulations it will be recognized that, in general, the water solubility of the salt is greater than the acid form. In solution form, the complex (or when desired the separate components) is dissolved in a physiologically acceptable carrier. Such carriers comprise a suitable solvent, preservatives such as benzyl alcohol, if needed, and physiologically compatible buffers. Useful solvents include, for example, water, aqueous alcohols, glycols, and phosphonate or carbonate esters. Such aqueous solutions contain no more than 50 percent of the organic solvent by volume. Examples of suitable buffers include the sodium, potassium or ammonium salts of weak acids, for example carbonates, phosphates, glycinates or arginates, N-methylglucosaminate or other amino acids, Tris, HEPES, MOPS, THAM or EPPS.

Injectable suspensions are compositions of the present invention that require a liquid suspending medium, with or without adjuvants, as a carrier. The suspending medium can be, for example, aqueous polyvinylpyrrolidone, inert oils such as vegetable oils or highly refined mineral oils, polyols, or aqueous carboxymethylcellulose. Suitable physiologically acceptable adjuvants, if necessary to keep the complex in suspension, may be chosen from among thickeners such as carboxymethylcellulose, polyvinylpyrrolidone, gelatin, and the alginates. Many surfactants are also useful as suspending agents, for example, lecithin, alkylphenol, polyethyleneoxide adducts, naphthalenesulfonates, alkylbenzenesulfonates, and polyoxyethylene sorbitan esters.

Isolated conformational isomers of bifunctional chelates based on CB-TE2A of the present invention can be produced by the synthetic scheme illustrated in Scheme 1. Alkylation of 1,4,8,11-tetraazabicyclo[6.6.2]hexadecane (1) with alkyl bromide derivative (2) results in monoalkylated macrocyclic derivative (3). Alkylation of (3) with bromo ester (4) produces a mixture of conformational isomers of the dialkylated macrocyclic derivative (5a, 5b). The most polar conformational isomer (5a) and the least polar conformational isomer (5b) can be isolated by a conventional isolation technique, such as reverse phase chromatography. Hydrolysis of isomer (5a) under basic conditions produces conformational isomer (6a) having rapid complexation kinetics with Cu(II) ions. Hydrolysis of isomer (5b) under basic conditions, however, produces conformational isomer (6b) having relatively slower complexation kinetics with Cu(II) ions than isomer (6a). Isomer (5a) can be converted to isomer (6b) under acidic hydrolysis conditions. Treatment of isomer (6b) with base does not result in the conversion of that isomer to isomer (6a) having rapid Cu(II) kinetics, however, isomer (5b) slowly converts to isomer (5a) in a polar solvent, a non-polar solvent, such as chloroform, or a mixture of polar and non-polar solvents that produce a homogeneous mixture of the conformational siomers. Selective hydrogenation of the nitro group in the resulting diacid (6a) using the method disclosed in Gowda et al. [26] produces bifunctional chelate (7a) having an amine group. The amine moiety of the bifunctional chelate can be converted to the bifunctional chelate (8a) having a isothiocyanate moiety following reaction with C(S)Cl₂ in CHCl₃.

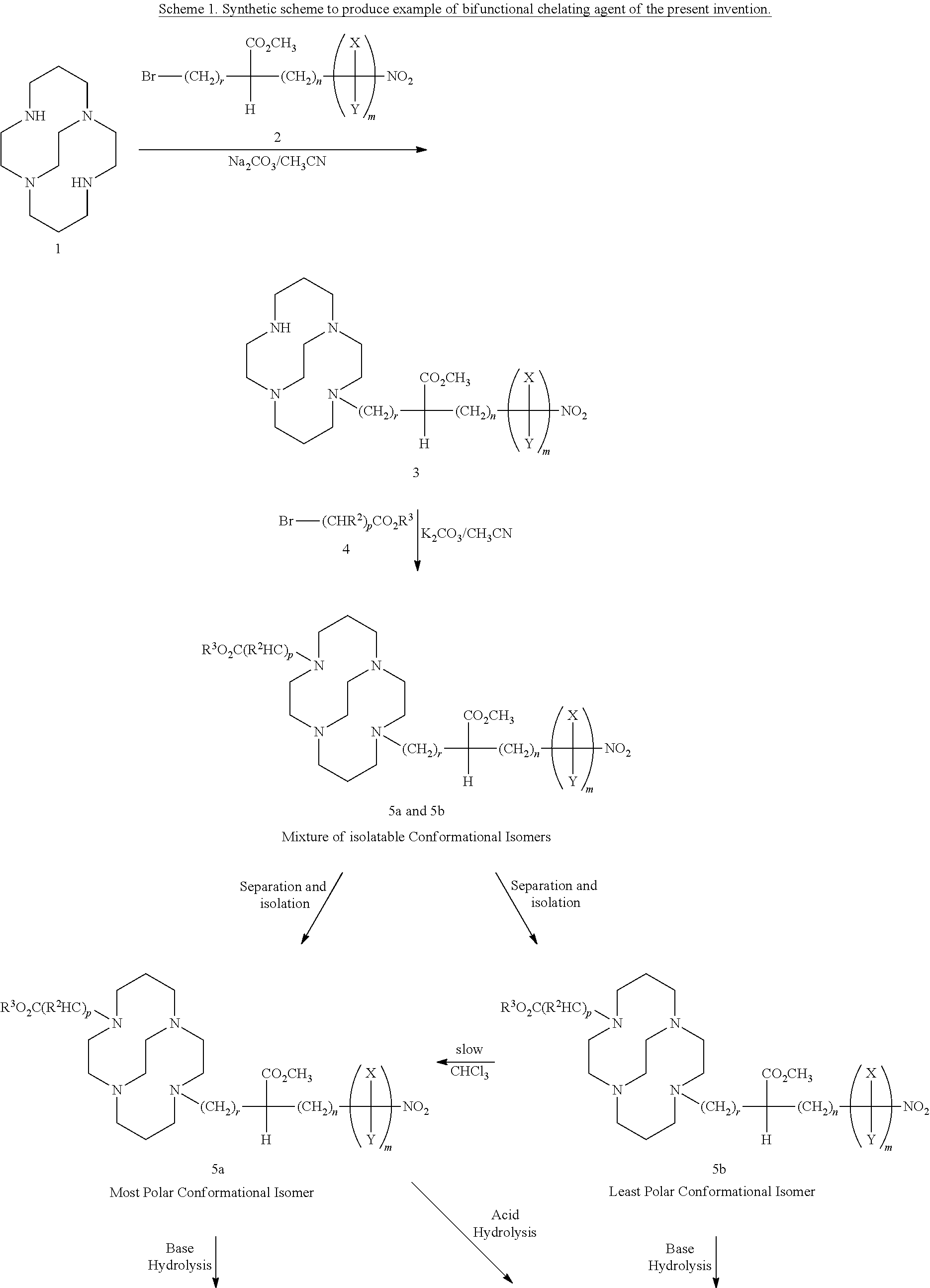

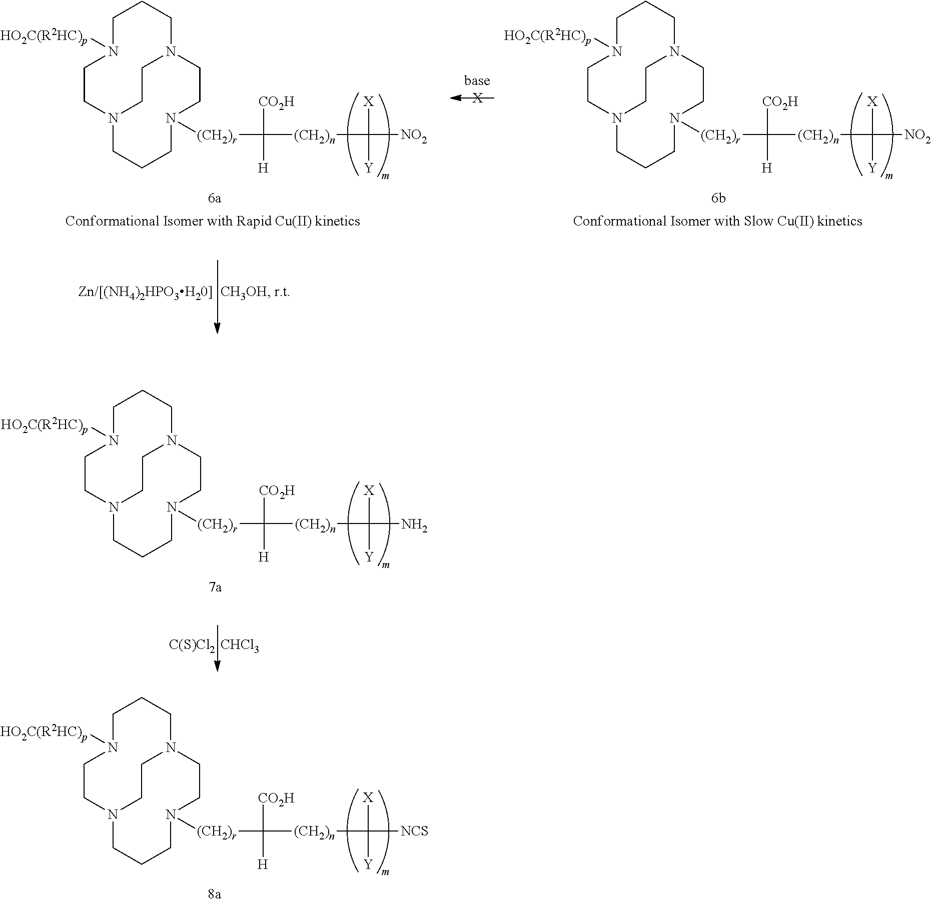

Although the synthetic schemes described above relate to the production of racemic ligands or chelators, it is to be understood that these schemes can be easily modified to produce enantiomerically pure or enantiomerically enriched ligands having the (L) or (D)-configuration by using enantiomerically pure or enantiomerically enriched starting materials, or by including one or more resolution steps within these schemes, which are generally known in the art.

As used herein, the terms “degree of complexation” and “percent complexation” are used interchangeably and are defined to mean the percentage of the ion that is successfully complexed with the bifunctional chelate. Here percent complexation is expressed as radiochemical yield, which is the yield of radiolabeled complex expressed as a fraction of the radioactivity originally present. The value of radiochemical yield obtained when making the ion complexes of the present reaction can be greater than 90% or greater than 95%, as measured by reverse phase chromatography (HPLC).

The conjugates of the present invention can be prepared by first forming the complex and then attaching to the biological carrier (pre-complexation). Thus, the process involves preparing or obtaining the ligand, forming the complex with an ion and then adding the biological carrier. Alternatively, the process may involve first conjugation of the ligand to the biological carrier and then the formation of the complex with an ion (post-complexation). Any suitable process that results in the formation of the ion-conjugates of this invention is within the scope of the present invention.

The complexes, bifunctional chelates and conjugates of the present invention are useful as diagnostic agents in the manner described. These formulations may be in kit form such that the two components (i.e., ligand and metal, complex and antibody, or ligand/antibody and metal) are mixed at the appropriate time prior to use. Whether premixed or as a kit, the formulations usually require a pharmaceutically acceptable carrier.

Tissue specificity may also be realized by ionic or covalent attachment of the chelate of formula (I) (where R⁶ is NH₂, isothiocyanato, semicarbazido, thiosemicarbazido, maleimido, bromoacetamido or carboxylic acid group) to a naturally occurring or synthetic molecule having specificity for a desired target tissue.

The following examples are provided to further illustrate the present invention, and should not be construed as limiting thereof.

### Example 1

### Synthesis of methyl 2-(1,4,8,11-tetraazabicyclo[6.6.2]hexadecan-4-yl)-4-(4-nitrophenyl)butanoate (10)

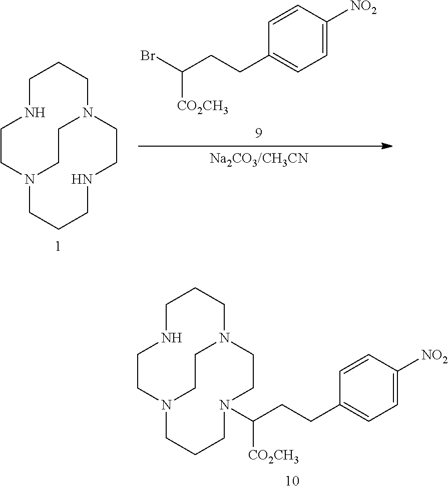

To an acetonitrile solution (80 mL) of CB-cyclam 1 (3 g, 13.2 mmol) was added a-bromo ester 9 (2.5 g, 8.2 mmol) and Na₂CO₃. The slurry was stirred at reflux for 18 hours. The reaction mixture was then cooled and filtered and the filtrate concentrated in vacuo. The crude product was dissolved in water (100 mL) and the pH adjusted to 3 using 6N HCl. The aqueous solution was then extracted with CHCl₃ (3×50 mL). The aqueous solution was then pH adjusted to 8 using 6N NaOH and re-extracted with CHCl₃ (3×50 mL) and the organic layer dried over anhydrous Na₂SO₄. The drying agent was filtered and the filtrate concentrated in vacuo to give 2.14 grams (63%) of 10 as a light-yellow oil. ¹H NMR (400 MHz, CDCl₃) δ 1.46 (m, 1H), 1.66 (m, 1H), 1.82 (m, 2H), 2.13 (q, 2H), 2.48 (m, 1H), 2.59-3.01 (m, 20H), 3.36 (t, 2H), 3.54 (m, 2H), 3.74 (s, 3H), 7.45 (d, 4-Ar, J_{H-H}=8.0 Hz, 2H), (d, J_{H-H}=8.0 Hz, 2H); ¹³C NMR (100 MHz, CDCl₃) δ 21.9, 27.2, 31.4, 32.9, 45.1, 48.2, 49.2, 51.4, 52.6, 52.8, 54.6, 54.9, 55.7, 55.9, 123.6, 129.5, 146.4, 149.3, 170.1.

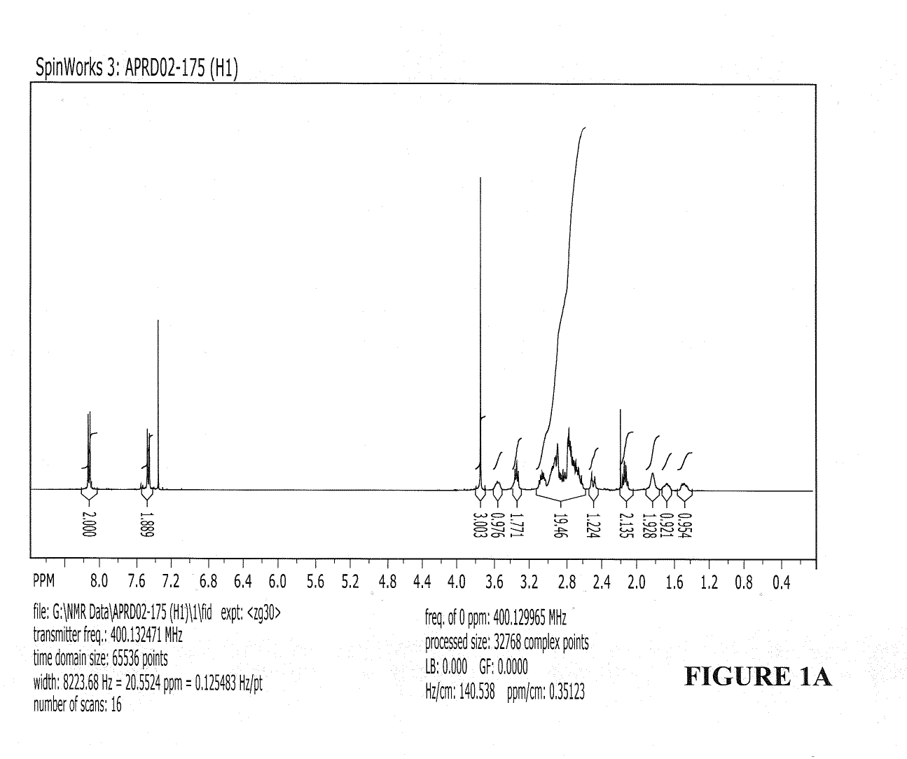

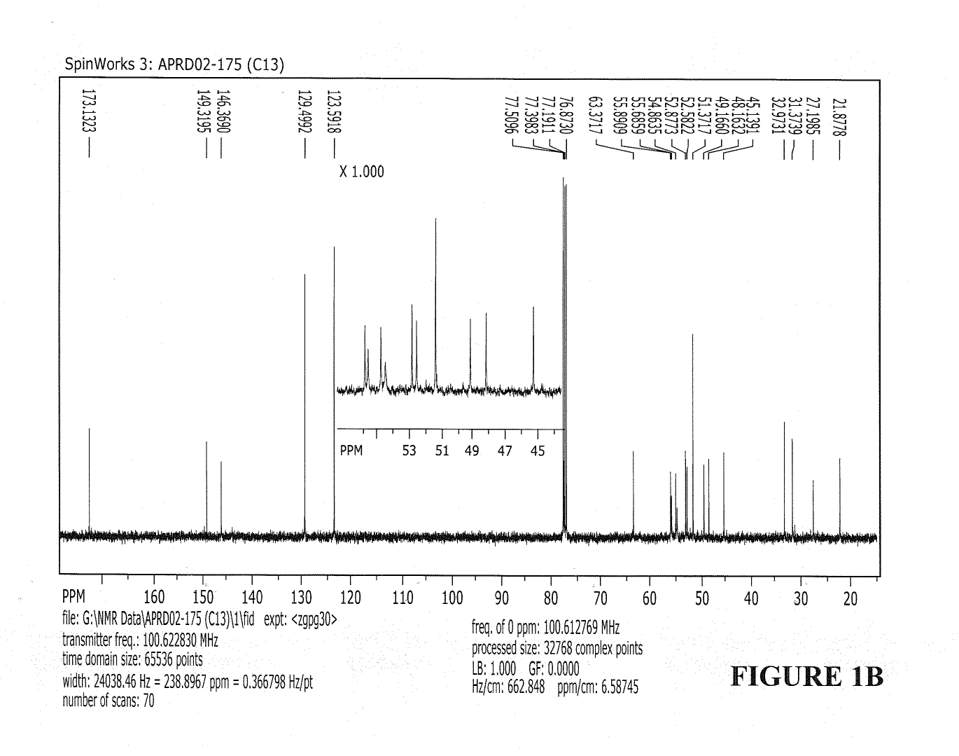

The ¹H-NMR and ¹³C-NMR spectra corresponding to the mono-alkylated product 10 are shown in FIGS. 1A and 1B, respectively.

### Example 2

### Synthesis of methyl 2-(11-(2-ethoxy-2-oxoethyl)-1,4,8,11-tetraazabicyclo[6.6.2]hexadecan-4-yl)-4-(4-nitrophenyl)butanoate

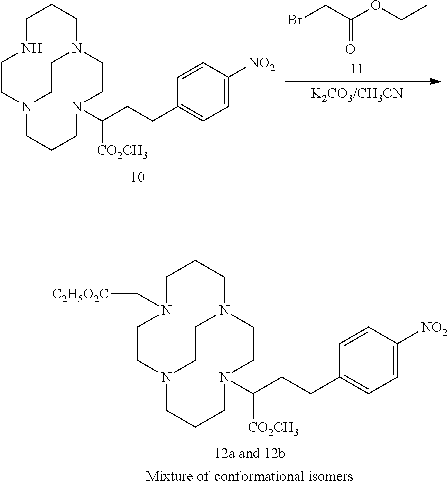

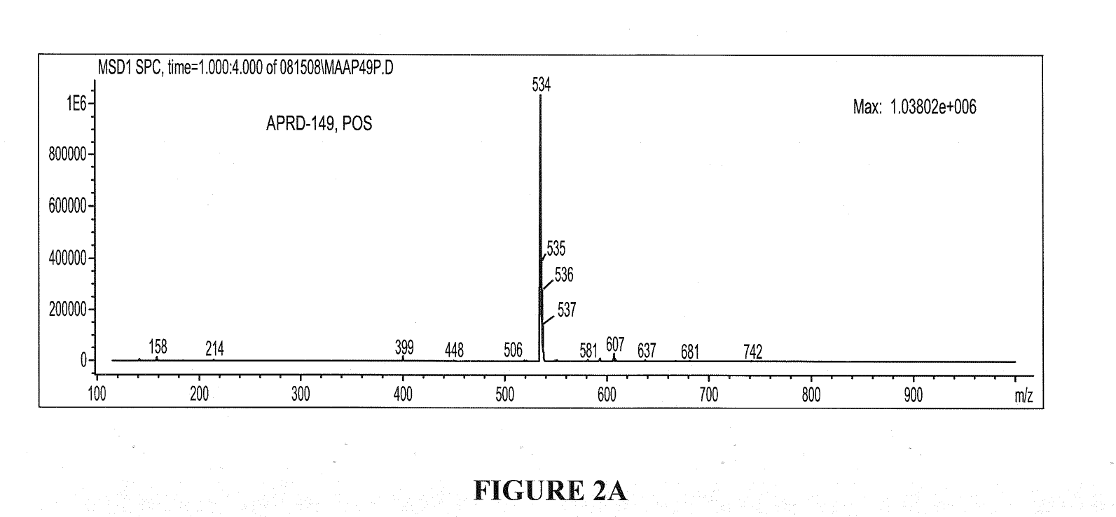

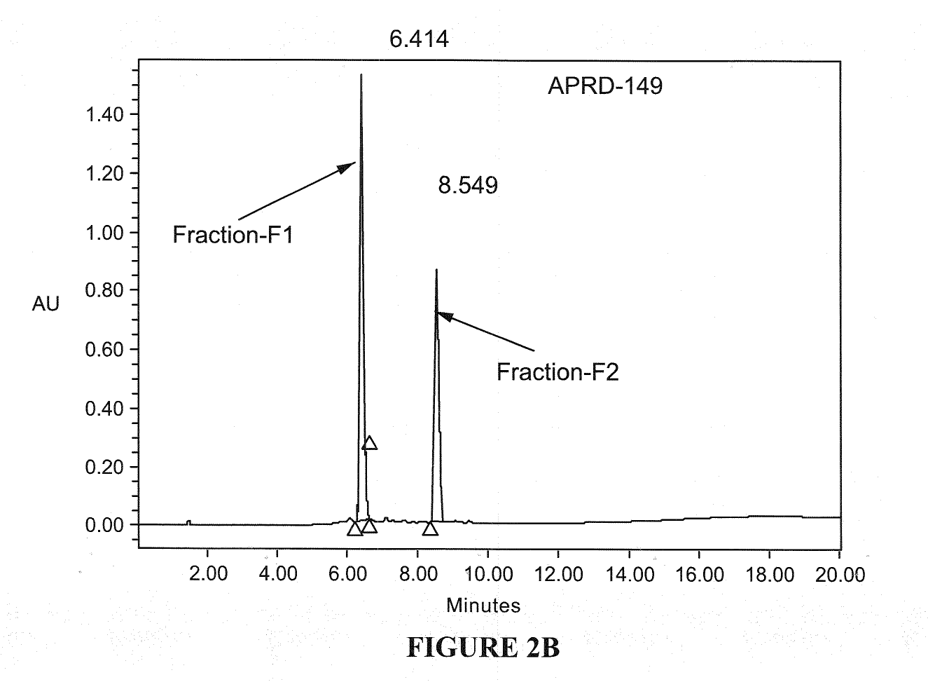

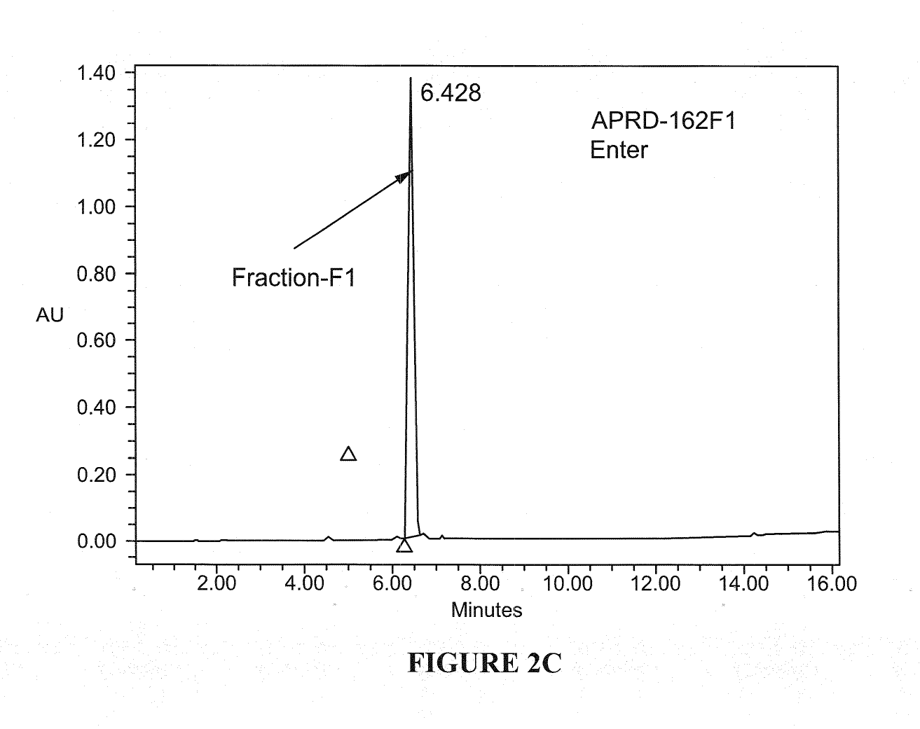

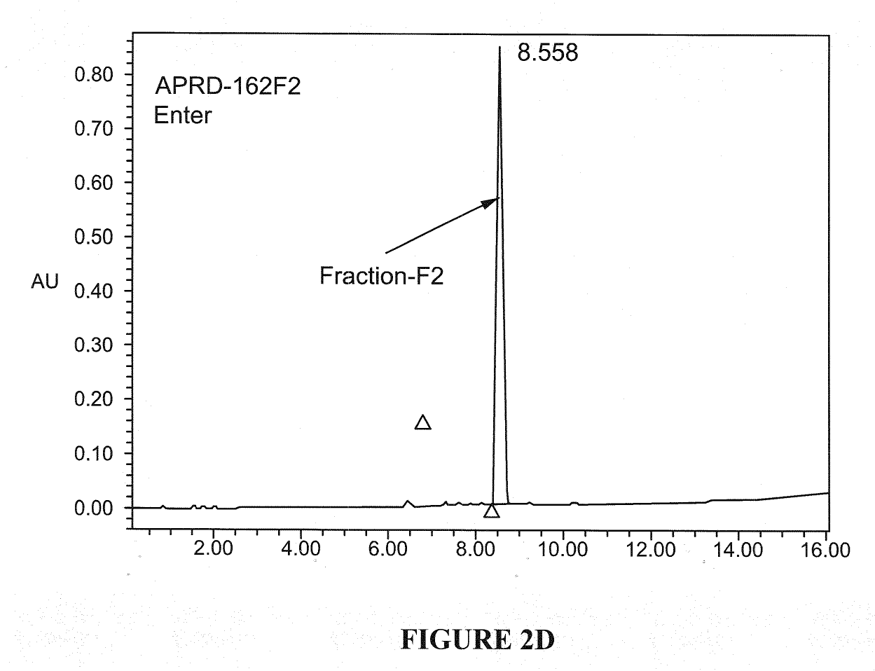

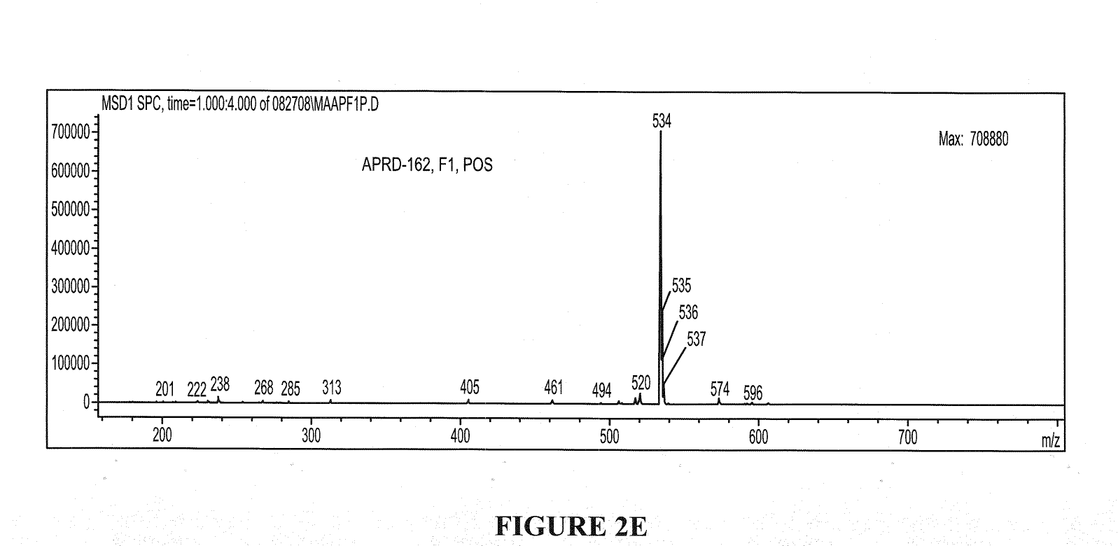

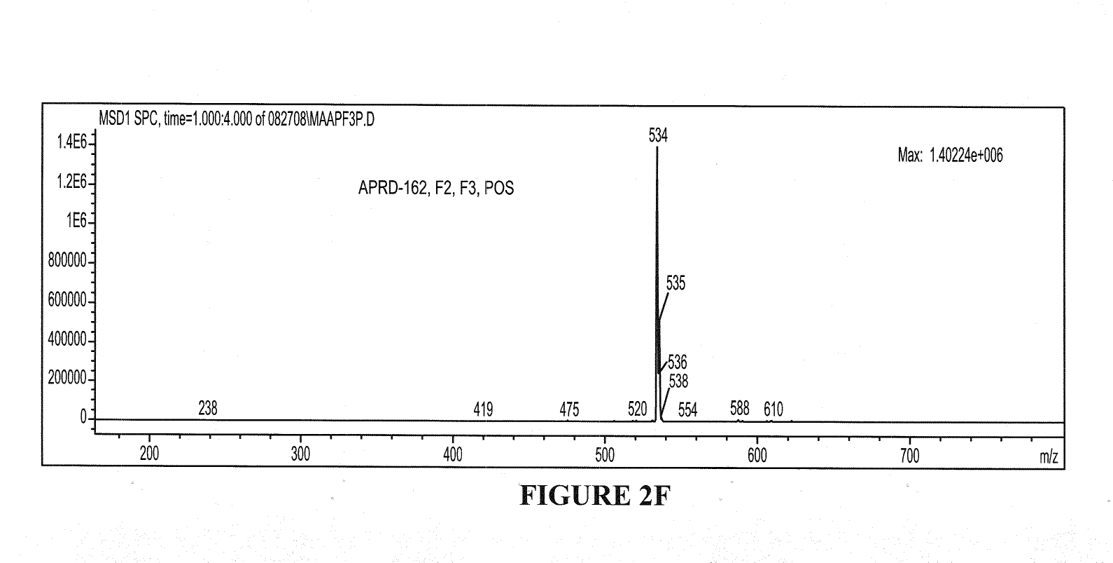

To an acetonitrile solution (80 mL) of 10 (2.1 g, 4.7 mmol) was added K₂CO₃ and the slurry stirred at −20° C. for 30 minutes. An acetonitrile solution (20 mL) of ethyl bromoacetate 11 (1.1 g, 6.6 mmol) cooled to −20° C. was then added and the reaction mixture stirred for 24 hours at −20° C. The reaction mixture was then warmed to 40° C. followed by stirring for an additional 12 hours. The reaction mixture was then filtered and the filtrate concentrated in vacuo and the crude product purified by column chromatography [silica, CHCl₃ (2% CH₃OH)]. The product 12a, 12b was isolated as a light-yellow solid (1.87 g, 75%). ESI mass spectrometry (positive-ion mode) produced a major signal m/s=534 corresponding to the desired product (FIG. 2A).

The product was evaluated by reverse phase HPLC using the following conditions:

### Column=Reztek Ultra IBD, 3μ

### 95% A/5% B−5% A/95% B

15 minute ramp

### A=H₂O (0.1% TFA)

### B=CH₃CN (0.1% TFA)

λ=225 nm
and surpisingly displayed two distinct species (F1 (12b) and F2 (12a); FIG. 2B).

The F1 and F2 components were purified from each other by reverse phase flash chromatography, and analysed by ¹H— and ¹³C-NMR.

Fraction F1: ¹H-NMR (400 MHz, CDCl₃) δ 1.19 (t, J_{H-H}=8.0 Hz, 3H), 1.24-1.44 (m, 4H), 1.73-2.01 (m, 2H), 2.13-2.74 (m, 18H), 2.83-3.00 (m, 2H), 3.03-3.26 (m, 4H), 3.64 (s, 4H), 4.07 (q, J_{H-H}=8.0 Hz, 2H), 7.27 (d, 4-Ar, J_{H-H}=8 Hz, 2H), 8.07 (d, 2-Ar, J_{H-H}=8 Hz, 2H); ¹³C-NMR (100 MHz, CDCl₃) δ 11.8, 24.8, 24.9, 26.4, 30.5, 47.5, 48.6, 48.8, 49.3, 50.0, 51.9, 52.5, 53.6, 53.9, 54.7, 54.8, 56.0, 57.5, 60.1, 121.1, 126.7, 143.9, 147.2, 169.8, 171.3. m/z: (ESI⁺); 534 (100% [M+H]⁺)
Fraction F2:'H-NMR (400 MHz, CDCl₃) δ 1.18 (t, J_{H-H}=7.2 Hz, 3H), 1.24-1.42 (m, 4H), 1.77-2.04 (m, 2H), 2.15-3.12 (m, 22H), 3.19-3.26 (m, 2H), 3.52 (dt, J=11.0 Hz, 5.0 Hz, 1H), 3.61 (s, 3H), 3.85 (dt, J=11.0 Hz, 5.0 Hz, 1H), 4.07 (q, J_{H-H}=7.2 Hz, 2H), 7.28 (d, 4-Ar, J_{H-H}=8.0 Hz, 2H), 8.07 (d, 2-Ar, J_{H-H}=8.0 Hz, 2H); ¹³C-NMR (100 MHz, CDCl₃) δ 11.8, 25.1, 25.2, 29.9, 30.6, 48.4, 48.5, 48.6, 49.0, 50.3, 50.8, 52.2, 53.8, 55.1, 56.5, 56.7, 57.6, 59.0, 121.2, 126.7, 143.9, 147.2, 169.8, 171.2. m/z: (ESI⁴); 534 (100% [M+H]⁺)

The isolated F1 and F2 components were also re-analysed by reverse phase HPLC to confirm that each component was isolated from the other (FIGS. 2C and 2D).

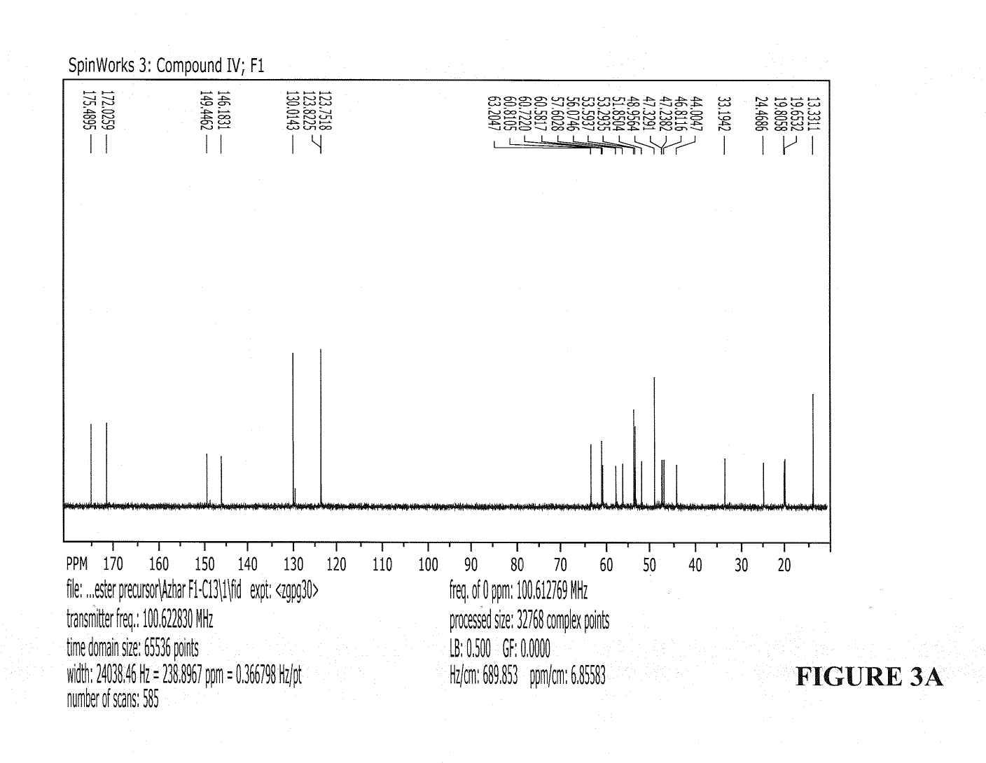

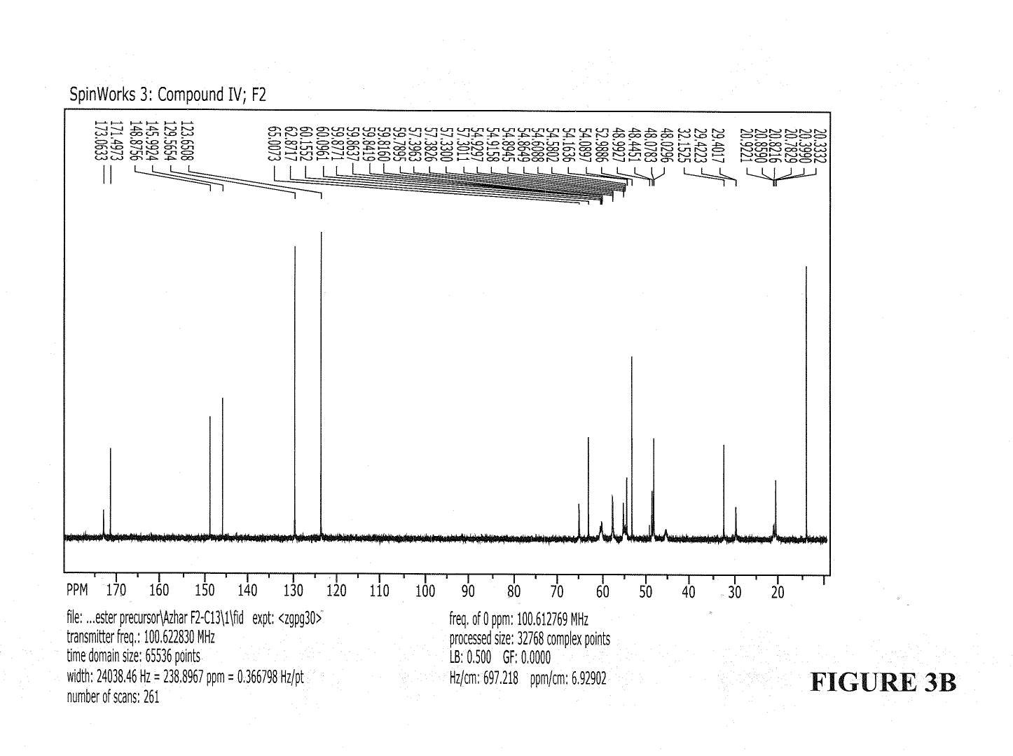

Each isolated component was re-evaluated by ESI mass spectromety and each was found to give identical patterns (FIGS. 2E and 2F). The ¹³C-NMR spectra for each component were, however, significantly different in the aliphatic region (FIGS. 3A and 3B).

### Example 3

### Acid Hydrolysis of methyl 2-(11-(2-ethoxy-2-oxoethyl)-1,4,8,11-tetraazabicyclo[6.6.2]hexadecan-4-yl)-4-(4-nitrophenyl)butanoate (12a)

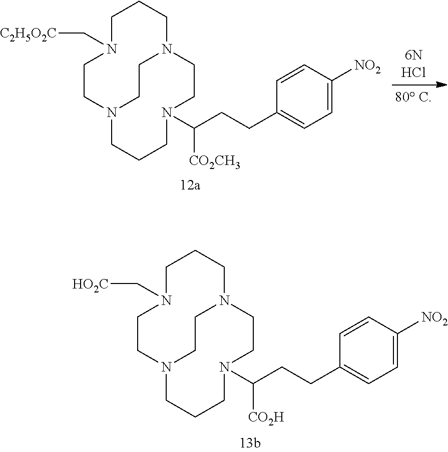

To an HCl solution (10 mL, 6M) was added 100 mg of 12a. The solution was heated to 80° C. with stirring for 12 hours, then cooled to room temperature and filtered through a 0.22 micron filter. The resulting filtrate was freeze-dried to give a white powder: ¹H-NMR (400 MHz, D₂O) δ 1.75 (m, 2H), 1.91-2.05 (m, 1H), 2.24-2.49 (m, 2H), 2.82-3.49 (m, 18H), 3.56-3.65 (m, 3H), 3.70 (d, J=10.0 Hz, 1H), 4.07 (d, J=17.0 Hz, 1H), 7.60 (d, 4-Ar, J_{H-H}=8.6 Hz, 2H), 8.25 (d, 2-Ar, J_{H-H}=8.6 Hz, 2H);

¹³C-NMR (100 MHz, D₂O) δ 18.9, 19.2, 24.8, 32.8, 44.5, 46.9, 47.3, 47.7, 52.4, 52.9, 53.2, 55.0, 58.0, 58.8, 58.9, 60.0, 123.9, 130.2, 146.3, 149.0, 171.6, 174.9.

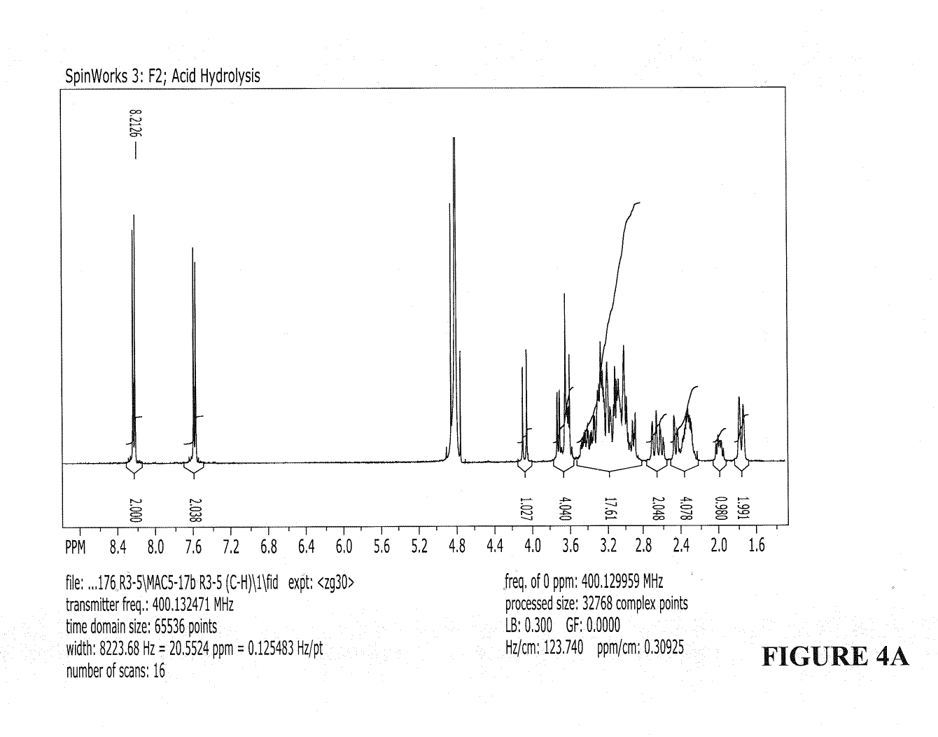

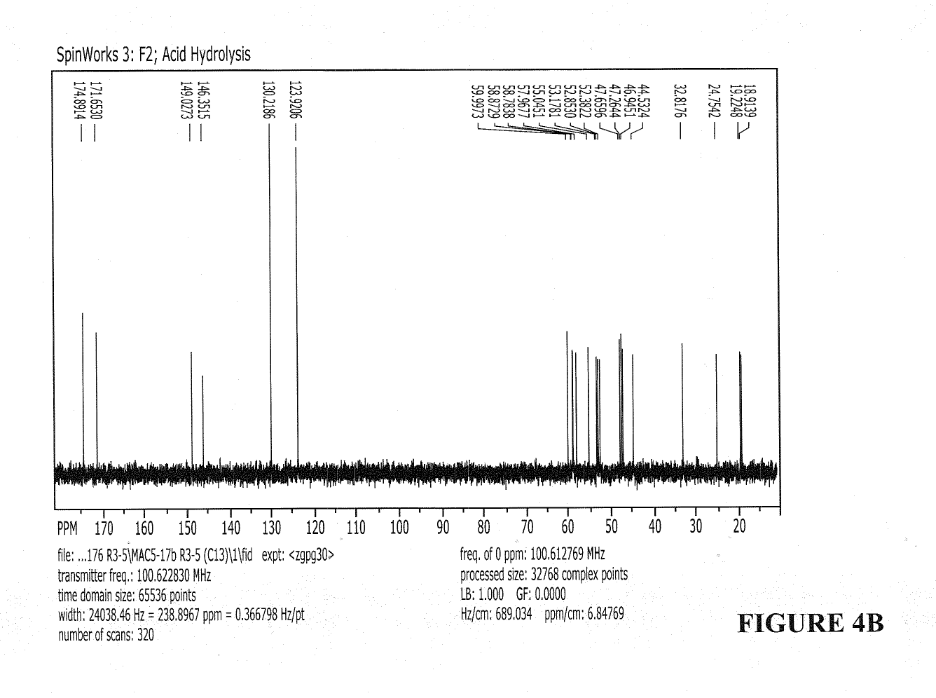

The ¹H-NMR and ¹³C-NMR spectra corresponding to the hydrolysed product 13b are shown in FIGS. 4A and 4B, respectively.

### Example 4

### Base Hydrolysis of methyl 2-(11-(2-ethoxy-2-oxoethyl)-1,4,8,11-tetraazabicyclo[6.6.2]hexadecan-4-yl)-4-(4-nitrophenyl)butanoate (12a)

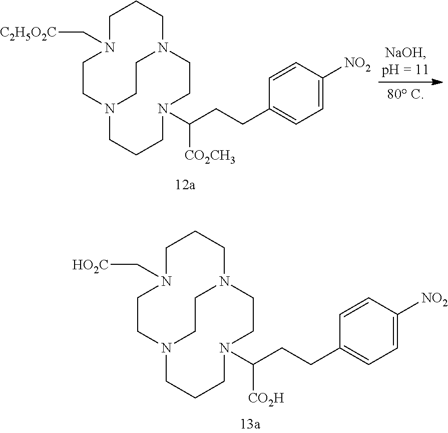

To an aqueous ethanol solution (40 mL, 1:1 H₂O/EtoH) adjusted to a value of pH of 12 with 6M NaOH was added 12a (200 mg) and the reaction mixture was maintained at a temperature 80° C. for 12 hours. The solution was cooled to room temperature and the pH adjusted to 7.2 with 1M HCl. The resulting solution was then concentrated in vacuo to give a white solid: ¹H-NMR (400 MHz, D₂O) δ 1.47-1.86 (m, 3H), 2.17-2.51 (m, 3H), 2.59-2.88 (m, 5H), 3.05-3.73 (m, 22H), 3.40-3.98 (m, 2H), 7.37 (d, 4-Ar, J=8.6 Hz, 2H), 8.06 (d, 2-Ar, J=8.6 Hz, 2H); ¹³C-NMR (100 MHz, D₂O) δ 19.4, 19.7, 46.3, 46.5, 47.1, 48.0, 52.2, 52.5, 52.6, 54.2, 56.6, 58.2, 58.9, 60.6, 62.6, 123.7, 129.4, 146.1, 148.7, 170.8, 172.2.

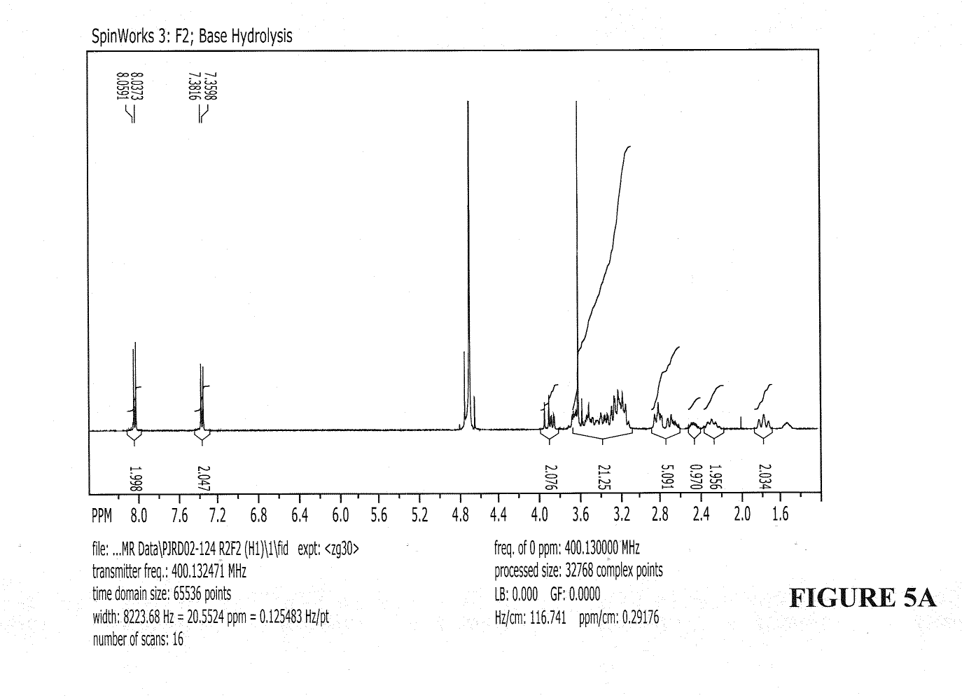

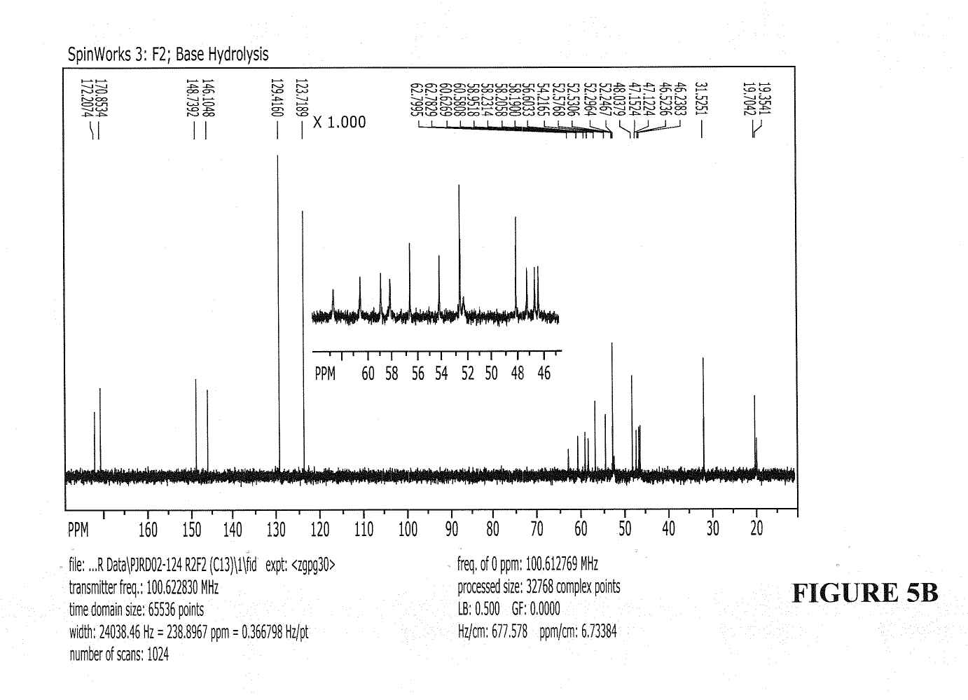

The ¹H-NMR and ¹³C-NMR spectra corresponding to the hydrolysed product 13a are shown in FIGS. 5A and 5B, respectively.

Both 13a and 13b were determined to have the same molecular ion by ESI-MS analysis. Furthermore, ¹H-¹³C COSY NMR analysis indicated that the two entities 13a and 13b were structurally equivalent with respect to bonds between atoms. It is therefore believed that 13a and 13b are conformational isomers of each other with 13a having a less rigid structure, which is supported by the differences observed in the ¹³C NMR spectra, the different retention times by high performance liquid chromatography (HPLC) and the strikingly different radiolabeling efficiency described below.

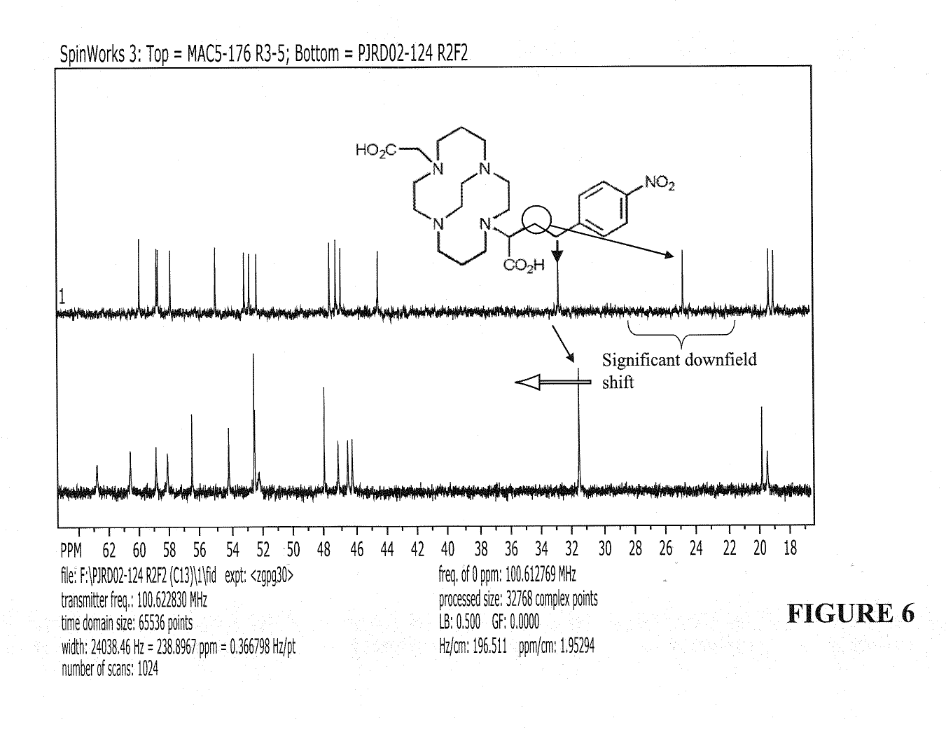

Comparison of the ¹³C NMR spectra of 13a and 13b clearly shows substantial differences between the two entities (FIG. 6). There are differences in the chemical shifts and the resonance intensities of 13a compared directly to 13b. The ¹³C NMR resonances in the aliphatic region for 13b have nearly equal intensity and similar T1 values (including the aliphatic carbons not confined in the macrocycle) indicating a highly rigid structure with low thermal motion. Contrastingly, the ¹³C NMR resonances of 13a show variable peak intensity reflecting greater rotational thermal motion and suggesting a less rigid structure compared to 13b.

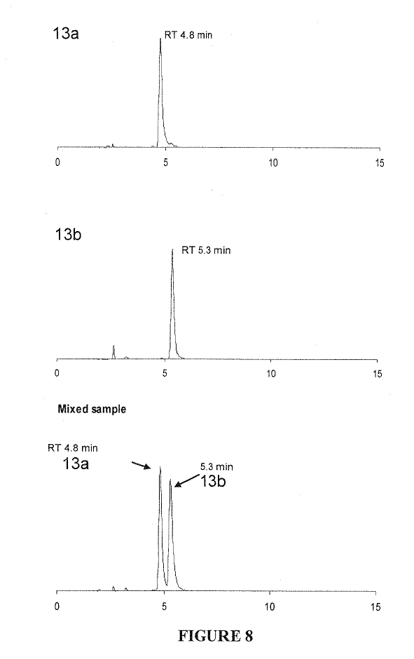

Reverse phase high performance liquid chromatography (HPLC) was done to further demonstrate the uniqueness of 13a and 13b. Three separate injections are shown in FIG. 8: 13a alone, 13b alone and a mixture of 13a and 13b. Injections of 13a and 13b showed that the two entities have different retention times, with 13a being slightly more polar than 13b. Injection of both samples together confirmed that 13a and 13b are unique entities which can be separated and resolved by reverse phase HPLC. The separations were conducted using a Waters X-Bridge BEH130 C18 column (4.6×150 mm) by isocratic elution using 20% acetonitrile/80% trifluoroacetic acid in water (0.01% v/v) at 1.0 mL/min.

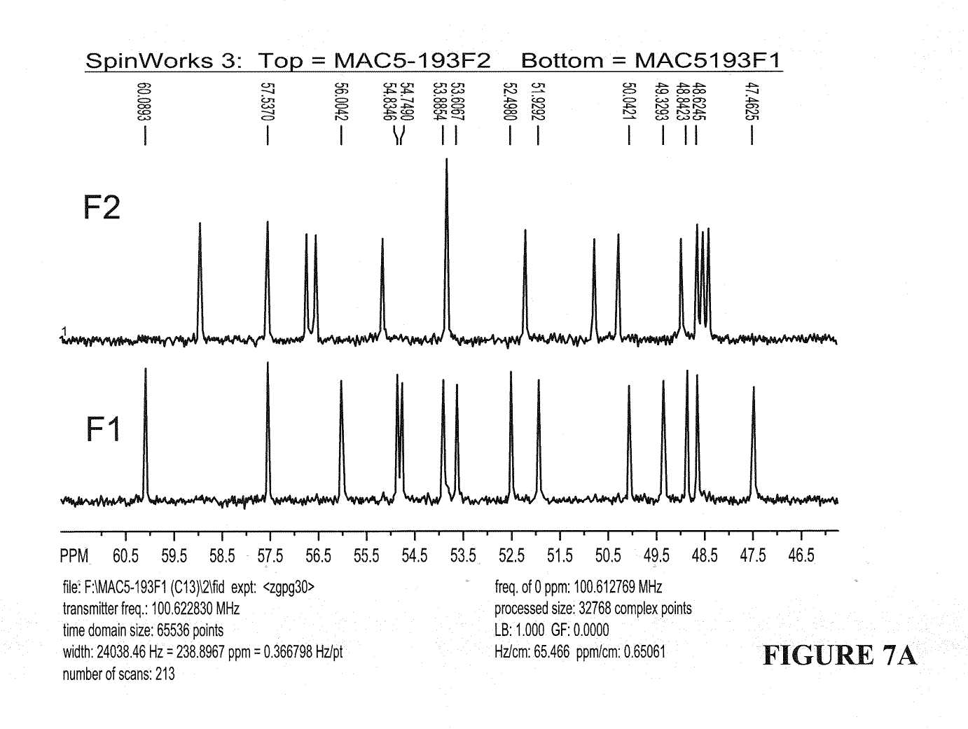

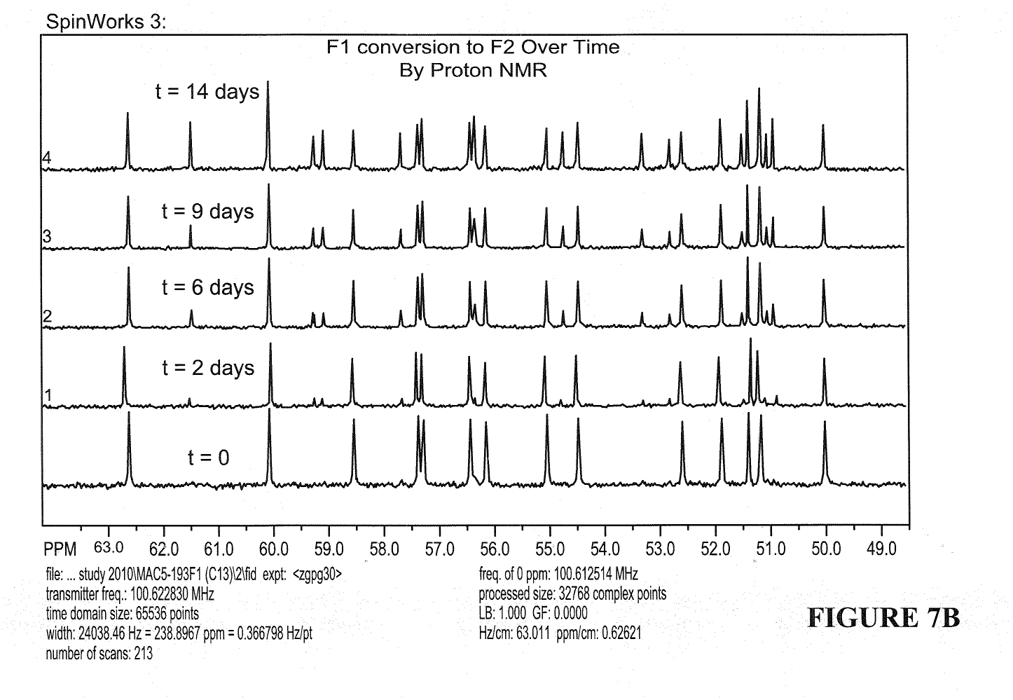

As illustrated in FIG. 7B, isomer 12b (F1) will slowly convert into isomer 12a (F2) if left in chloroform for an extended period of time. After 14 days at room temperature, the conversion 12b (F1) to 12a (F2) was approximately 50% complete.

### Example 5

### Preparation of 2-(11-(carboxymethyl)-1,4,8,11-tetraazabicyclo[6.6.2]hexadecan-4-yl)-4-(4-isothiocyanatophenyl)butanoic acid (Conformational Isomer 15b)

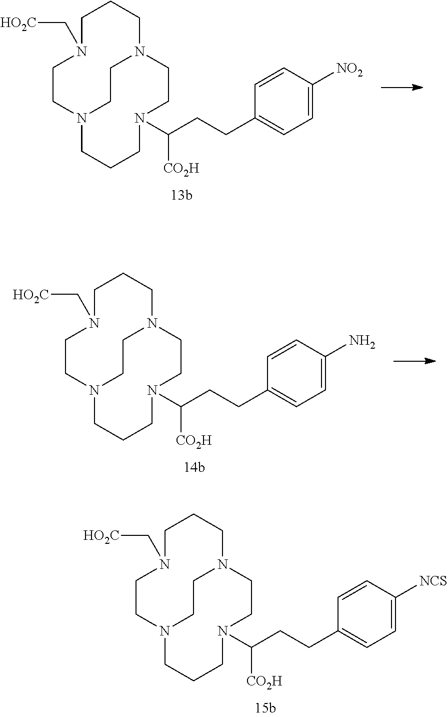

To an aqueous solution of 13b (50 mg; 10 mL water) was added 10% Pd/C (25 mg). The suspension was then the was pressurized to 30 psi H₂ for two hours in a Paar hydrogenator. The solution was then purged with argon for 10 minutes then filtered. The aqueous filtrate containing amine 14b was added to CHCl₃(10 mL) along with 50 μL of thiophosgene and the resulting solution was vigorously stirred for one hour. The aqueous layer was then separated and washed with water (3×10 mL) and the aqueous layer freeze-dried to provide 15b as a light yellow solid.

### Example 6

### Preparation of 2-(11-(carboxymethyl)-1,4,8,11-tetraazabicyclo[6.6.2]hexadecan-4-yl)-4-(4-isothiocyanatophenyl)butanoic acid (Conformational Isomer 15a)

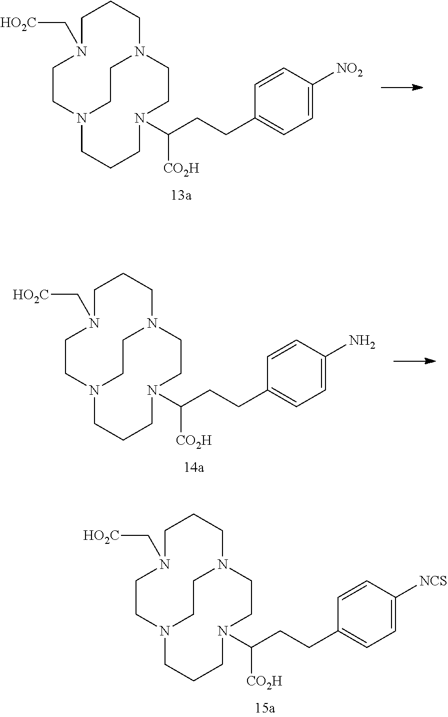

To an aqueous solution of 13a (50 mg; 10 mL water) was added 10% Pd/C (25 mg). The suspension was then pressurized to 30 psi H₂ for 2 hours in a Paar hydrogenator. The solution was then purged with argon for 10 minutes then filtered. The aqueous filtrate containing amine 14a was added to CHCl₃ (10 mL) along with 50 μL of thiophosgene and the resulting solution was vigorously stirred for 1 hour. The aqueous layer was then separated and washed with water (3×10 mL) and the aqueous layer freeze-dried to provide 15a as a light yellow solid.

### Example 7

### Comparison of the Radiolabeling Conditions and Efficiency of 13a and 13b with a Cu Radioisotope Applicable to Nuclear Imaging and Therapy

The conditions required to radiolabel 13a and 13b with Cu-64 in >95% radiochemical yield were optimized for pH, temperature and reaction time (Table 1). The exact same radioisotope lot, buffer solutions, and conditions were used for the reaction and the analysis of both 13a and 13b.

TABLE 1
Optimized reaction conditions for Cu-64 radiolabeling
of 13a and 13b to >95% radiochemical yield

| Conditions | 13a | 13b |
|---|---|---|
| Temperature | Room temperature (~20° C.) | 80° C. |
| pH | 5-6 (10 mM sodium acetate | 6 (10 mM sodium acetate |
| | buffer) | buffer) |
| CB-TE2A BFC | As low as 6 μM | 50 μM or higher |
| (6a/6b) | | |
| Concentration | | |
| Reaction time | 10-30 minutes depending on | 60 minutes |
| | CB-TE2A BFC concentration | |

The radiolabeling conditions for 13a are far superior to 13b with greater application in radiopharmaceutical development. The room temperature radiolabeling conditions for 13a make it applicable to radiolabeling heat-sensitive biomolecules such as antibodies and proteins. The low concentration of 13a required for efficient Cu-64 radiolabeling facilitates the development of high specific activity radiopharmaceuticals, which would have better targeting properties (i.e. better tumour- or disease-state targeting properties compared to a lower specific activity agent). Finally, the shorter reaction times for radiolabeling 13a is preferred to limit loss of radioisotope to decay and to facilitate less time-consuming preparation in a radiopharmacy or hospital setting.

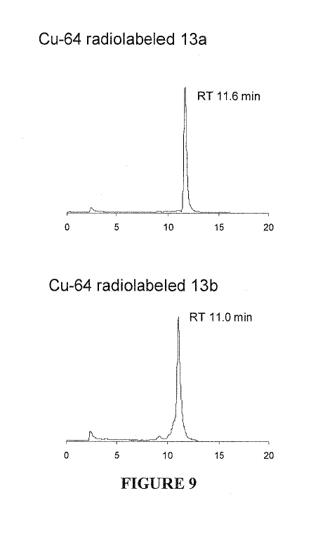

Reverse phase HPLC analysis of Cu-64 radiolabeled 13a and 13b is shown in FIG. 9. Cu-64 radiolabeled 13a and 13b are distinct species with different retention times by HPLC. The analysis was conducted with a Phenomenex hydrosynergy RPC18 column (4.6×150 mm) using a gradient of 98% trifluoracetic acid in water (0.01% v/v) 2% acetonitrile to 50% trifluoracetic acid in water (0.01% v/v) 50% acetonitrile over 30 min at 1 mL/min.

Since previously reported derivatives of CB-TE2A require harsh (high temperature and basic pH) conditions for radiolabeling [20-22], they are not applicable to Cu radiolabeling and imaging with sensitive biomolecules such as antibodies. To illustrate the utility of the CB-TE2A conformational isomers of the present invention, which do allow for mild room temperature Cu radiolabeling, a proof-of-principle antibody labeling and small animal imaging study were performed.

### Example 8

### Antibody Radiolabeling

Conjugation of the antibody trastuzumab and isothiocyanate derivatives 15a and 15b were each separately conjugated to trastuzumab, an antibody with affinity for the HER2/neu receptor. The isothiocyanate derivatives 15a and 15b (1.5 mg) were dissolved in water (200 μL). The resulting solutions (50 μL, 0.375 mg, 0.456 mmol) were then each added to a mixture of trastuzumab aqueous solution (1000 μL, 22 mg, 0.152 μmol) and HEPES buffer (1450 μL, 50 mM, pH 3.5). The resulting reaction mixtures were vortexed and then allowed to react at room temperature for 18 hours. The mixtures were purified using a PD-10 size exclusion column (Sephadex G-25, GE Healthcare), which had been conditioned with 10 mM sodium acetate buffer, pH 5, by loading the entire 2.5 mL solution on the column and eluting with sodium acetate buffer (3.5 mL). The average number of chelates attached per antibody was determined to be 0.7-0.9.

### Radiolabeling of the Antibody Conjugates

The resulting antibody conjugates were radiolabeled with Cu-64. Cu-64 (2 mCi) was added to one of the antibody conjugates in 10 mM sodium acetate buffer (pH 5.5 or 6). Radiolabeling was monitored using size exclusion chromatography. The conditions and results of the radiolabeling reaction are summarized in Table 2.

TABLE 2
Radiolabeling optimization conditions and labeling yield for both CB-TE2A conjugates

| | | Cu-64 Activity | | | Labeling |
|---|---|---|---|---|---|
| Conjugate | Amount | (mCi) | Buffer | Conditions | Yield |
| CBTE2A*HER | 0.5 mg | ~2 | 10 mM NaOAc pH 5.5 | RT, 30 min | 48.0% |
| (15a•HER) | | | | | |
| CBTE2A*HER | 0.5 mg | ~2 | 10 mM NaOAc pH 6 | RT, 30 min | 50.5% |
| (15a•HER) | | | | | |
| CBTE2A*HER | 0.5 mg | ~2 | 10 mM NaOAc pH 5.5 | RT, 60 min | 54.0% |
| (15a•HER) | | | | | |
| CBTE2AHER | 0.5 mg | ~2 | 10 mM NaOAc pH 6 | RT, 30 min | 7.0% |
| (15b•HER) | | | | | |
| CBTE2AHER | 0.5 mg | ~2 | 10 mM NaOAc pH 6 | 80° C., 60 min | 6.5% |
| (15b•HER) | | | | | |
| CBTE2AHER | 1.0 mg | ~2 | 10 mM NaOAc pH 6 | 80° C., 60 min | 0.0% |
| (15b•HER) | | | | | |
| CBTE2AHER | 0.5 mg | ~2 | 10 mM NaOAc pH 6 | 40° C., 60 min | 41.5% |
| (15b•HER) | | | | | |

Radiolabeling of the antibody conjugates again illustrates the significant kinetic differences between the two isolated conformations of the bifunctional CB-TE2A derivatives. For the fast labeling conformation of CB-TE2A antibody conjugate (15a•HER), reasonable radiochemical yields could be achieved at room temperature within 30 min at pH 5.5-6. For the slow labeling conformation of CB-TE2A antibody conjugate (15b•HER), negligible yields were obtained at room temperature. At 80° C., the conjugate 15b•HER decomposes so no radiolabeled product was obtained. At slightly elevated temperature and longer reaction times, the slow labeling conformation of CB-TE2A antibody conjugate (15b•HER) could be labeled with Cu-64, but with lower radiochemical yield than the superior fast labeling conformation.

### Example 9

### Tumour Imaging with Cu-64 Radiolabeled Antibody Conjugates

### Cell Lines

The human breast cancer line, MDA-MB-435, was transfected with an empty vector (LCC6^{Vector}) or one containing the HER-2/neu gene (LCC6^{HER-2}) previously at the BC Cancer Agency.[27] The LCC6^{Vector} and LCC6^{HER-2} cells have low and high expression levels of the HER-2 receptor, respectively, and both cell lines are tumourigenic forming tumours robustly in Rag2M mice within 2-3 weeks[27-28]. The two cell lines were maintained in DMEM supplemented with 2 mM L-glutamine (StemCell Technologies, Vancouver, BC) and 10% fetal bovine serum (FBS) (HyClone, Logan, Utah). Frozen cells contained G418 (500 μg/ml, Mediatech, Inc., Herndon, Va.). Cells were expanded in DMEM-10% FBS, with no G418, for at least 3 passages before expansion for animal studies. Prior to use, cells were detached from the surface of the tissue culture flask by treatment with 0.25% Trypsin/EDTA.

### PET Imaging and Biodistribution Studies

HER-2 negative (LCC6^{Vector}) and positive (LCC6^{HER-2}) tumours were grown subcutaneously on Rag2M mice; briefly, 5×10⁶ cells (50 uL) were injected subcutaneously on the lower back of Rag2M mice. Tumour volumes were measured using calipers and calculated from 2 orthogonal dimensions using the formula, (π/6×length×width). When tumour volumes reached ˜150 mm³, mice were injected intravenously with the Cu-64 radiolabeled trastuzumab conjugated to either of the CB-TE2A conformational isomers (5.2-5.9 MBq, 25-40 GBq/μmol). Groups (n=4) of mice (LCC6^{Vector} and LCC6^{HER-2}) were injected intravenously through the tail vein and were imaged at ˜28 and ˜44 h post-injection. Imaging was carried out in the Siemens Inveon multi-modality CT-PET small animal scanner. PET data were acquired in list mode acquisition (20 minutes) and subsequently histogrammed in a single frame. CT-based attenuation scans to correct for the animal's body mass were carried out immediately before each PET scan. PET images were reconstructed in 3D using OSEM-MAP3D algorithms supplied by Siemens. Once imaged, mice were euthanized, the blood, liver, kidney, muscle and tumour were harvested, weighed and placed in a gamma counter to determine the activity present per gram tissue.

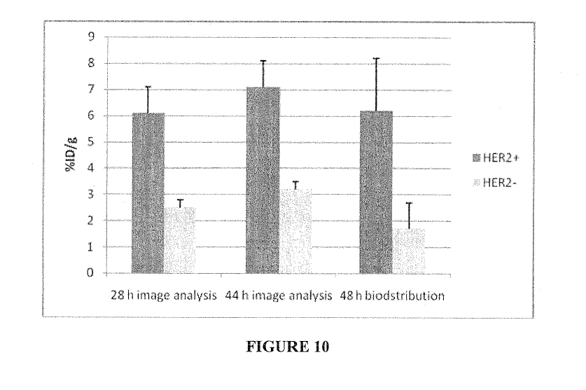

Imaging and biodistribution results for the two Cu-64 radiolabeled antibody conjugates of the CB-TE2A isomers were statistically similar. For both, uptake in the Her2/neu positive tumour was significantly higher than uptake in the Her2/neu negative tumour, illustrating the similarity of these bifunctional chelates for antibody imaging (see FIG. 10). As well, high levels of activity were observed in the blood circulation and liver, similar to other radiolabeled antibodies.[29]

All citations are hereby incorporated by reference.

### REFERENCES

1. Williams, H. A., et al., A comparison of PET imaging characteristics of various copper radioisotopes. Eur. J. Nucl. Med. Biol., 2005. 32: p. 1473-1480.
2. Bryan, J. N., et al., Comparative uptakes and biodistribution of internalizing vs. moninternalizing copper-64 radioimmunoconjugates in cell and animal models of colon cancer. Nucl. Med. Biol., 2005. 32: p. 851-858.
3. Li, M. and C. F. Meares, Synthesis, metal chelate stability studies, and enzyme digestion of a peptide-linked DOTA derivtive and its corresponding radiolabeled immunoconjugates. Bioconjugate Chem., 1993.4: p. 275-283.
4. Moshin, H., et al., Preparation and biological evaluation of ¹¹¹In-, ¹⁷⁷ Lu- and ⁹⁰Y labeled DOTA analogues conjugated to B72.3. Nucl. Med. Biol., 2007. 34: p. 493-502.
5. Liu, S., et al., Comparison of yttrium and indium complexes of DOTA-BA and DOTA-MBA: Models for ⁹⁰Y- and ¹¹¹In-labeled DOTA-biomolecule conjugates. Bioconjugate Chem., 2002. 13: p. 902-913.
6. Carrasquillo, J. A., et al., Similarities and differences in ¹¹¹In- and ⁹⁰Y-labeled 1B4M-DTPA antiTac monoclonal antibody distribution. J. Nucl. Med., 1999. 40: p. 268-276.
7. Cole, W. C., et al., Comparative serum stability of radiochelates for antibody radiopharmaceuticals. J. Nucl. Med., 1987. 28: p. 83-90.
8. Cole, W. C., et al., Serum stability of ⁶⁷ Cu chelates: comparison with In and ⁵⁷Co. Nucl. Med. Biol., 1986. 13: p. 363-368.
9. Jones-Wilson, T. M., et al., The in vivo behaviour of copper-64-labeled azamacrocyclic complexes. Nucl. Med. Biol., 1998. 25: p. 523-530.
10. De Leon-Rodriguez, L. M. and Z. Kovacs, The synthesis and chelation chemistry of DOTA-peptide conjugates. Bioconjugate Chem., 2008. 19: p. 391-402.
11. Boswell, C. A., et al., Comparative in vivo stability of copper-64-labeled cross-bridged and conventional tetraazamacrocyclic complexes. J. Med. Chem., 2004. 47: p. 1465-1474.
12. Zhang, K., et al., Vascoactive intestinal peptide (VIP) and pituitary adenylate cyclase activating peptide analogues for PET imaging of breast cancer: In vitro/in vivo evaluation. Regul. Pept., 2007. 144: p. 91-100.
13. Di Bartolo, N. M., et al., Synthesis of a new cage ligand, SarAr, and its complexation with selected transition metal ions for potential use in radioimaging. J. Chem. Soc., Dalton Trans., 2001: p. 2303-2309.
14. Ferreira, C. L., et al., Evaluation of novel bifunctional chelates for the development of Cu-64-based radiopharmaceuticals. Nucl. Med. Biol., 2008. 35(8): p. 875-882.
15. Brogan, J. B., Multifunctional cross-bridged tetraza macrocyclic compounds and methods of making and using. 2006, General Electric Company: US.
16. Weisman, G. R., et al., Cross-bridged cyclam. Protonation and lithium cation (Li⁺) complexation in a diamond-lattice cleft. J. Am. Chem. Soc., 1990. 112(23): p. 8604-8605.
17. Weisman, G. R., et al., Synthesis and transition-metal complexes of new cross-bridged tetraamine ligands. Chem. Commun., 1996: p. 947-948.
18. Sun, X., et al., Radiolabeleing and in vivo behaviour of copper-64-labeled cross-bridged cyclam ligands. J. Med. Chem., 2002. 45: p. 469-477.
19. Sprague, J. E., et al., Synthesis, characterization and in vivo studies of Cu-(II)-64-labeled cross-bridged tetraazamacrocycle-amide complexes as models of peptide conjugate imaging agents. J. Med. Chem., 2007. 50: p. 2527-2535.
20. Wadas, T. J. and C. J. Anderson, Radiolabeling of TETA- and CB-TE2A-conjugated peptides with copper-64. Nature Protocols, 2006. 1(6): p. 3062-3068.
21. Boswell, C. A., et al., Optimization of labeling and metabolite analysis of copper-64-labeled azamacrocylic chelators by radio-LC-MS. Nucl. Med. Biol., 2005. 32: p. 29-38.
22. Boswell, C. A., et al., Comparative in vivo stability of coppers-64-labeled cross-bridged and conventional tetraazamacrocyclic complexes. J. Med. Chem., 2004. 47: p. 1465-1474.
23. Wadas T. J., et al., Radiolabelling of TETA- and CB-TE2A-conjugated peptides with copper-64. Nature Protocols, 2006, 1: p. 3062-3068.
24. Boswell, C. A., et al., Synthesis of a Cross-Bridged Cyclam Derivative for peptide Conjugation and ⁶⁴Cu Radiolabelling. Bioconjugate Chem., 2008, 19: p. 1476-1484.
25. Sprague, J. E., et al., Synthesis, Characterization and In Vivo Studies of Cu(II)-64-Labelled Cross-Bridged Tetrazamacrocyle-amide Complexes as Models of Peptide Conjugate Imagaing Agents. J. Med. Chem., 2007. 30: p. 2527-2535.
26. Gowda, D. C., et al., Chemoselective Hydrogenation of Aromatic Nitro Compounds Using Diammonium Hydrogen Phosphite and Commercial Zinc Dust. E-J. Chem., 2008. 5(4): p. 914-917.
27. W. H. Dragowska, C. Warburton, D. T. T. Yapp, A. I. Minchinton, Y. Hu, D. N. Waterhouse, K. Gelmon, K. Skov, J. Woo, D. Masin, L. A. Huxman, A. H. Kyle and M. B. Bally, Mol. Cancer. Res. 2004, 2, 606-619.
28. C. L. Ferreira, D. T. T. Yapp, S. Crisp, B. W. Sutherland, S. S. W. Ng, M. Gleave, C. Bensimon, P. Jurek and G. E. Kiefer, Eur. J. Nucl. Med. Mol. Imaging 2010, 37, 2117-2126.
29. a) U. Elasser-Beile, G. Reischl, S. Wiehr, P. Buhler, P. Wolf, K. Alt, J. E. Shively, M. S. Judenhofer, H.-J. Machulla and B. J. Pichler, J. Nucl. Med. 2009, 50, 606-611; b) G. Niu, Z. Li, Q. Cao and X. Chen, Eur. J. Nucl. Med. Mol. Imaging. 2009, 36, 1510-1519.

1. An isolated conformational isomer of a bifunctional chelating agent of formula (I): 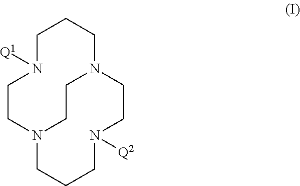
wherein:
Q¹ is —(CHR²)ₚCO₂R³ or —(CHR²)ₚPO₃R⁴R⁵;
Q² is 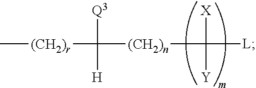
Q³ is —(CHR²)_{w}CO₂R³ or —(CHR²)_{w}PO₃R⁴R⁵;
each R² is independently hydrogen; C₁-C₄ alkyl or (C₁-C₂alkyl)phenyl;
each R³ is independently H, benzyl, C₁-C₄ alkyl, or a protecting group;
R⁴ and R⁵ are independently H, C₁-C₆ alkyl, (C₁-C₂ alkyl)phenyl or a protecting group;
X and Y are each independently hydrogen or may be taken with an adjacent X and Y to form an additional carbon-carbon bond;
m is an integer from 0 to 5 inclusive;
n is an integer from 1 to 5 inclusive;
p is 1 or 2;
r is 0 or 1;
w is 0 or 1;
L is a linker/spacer group covalently bonded to a carbon atom and replaces one hydrogen atom of said carbon atom, said linker/spacer group being represented by the formula: 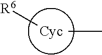
wherein:
R⁶ is H or an electrophilic, nucleophilic or electron-rich moiety which allows for covalent attachment to a biological carrier, or synthetic linker which can be attached to a biological carrier, a protected form thereof or a precursor thereof; and
Cyc represents a linear, branched or cyclic aliphatic moiety, aromatic moiety, aliphatic heterocyclic moiety, or aromatic heterocyclic moiety, each of said moieties optionally substituted with one or more groups which do not interfere with binding to a biological carrier;
or a pharmaceutically acceptable salt thereof,
wherein the conformational isomer is prepared by a process comprising:
a) reacting a compound of formula: 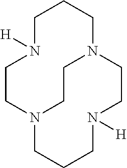
with a compound of formula Q²-LG, wherein Q² is as defined above and LG is a leaving group, to form a mixture of conformational isomers of a mono-alkylated derivative of formula: 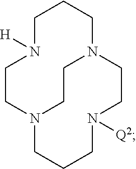
b) reacting the mixture of conformational isomers of the monoalkylated derivative with a compound of formula Q¹-LG, wherein Q¹ is as defined above and LG is a leaving group, to form a mixture of conformational isomers of the compound of formula (I), the mixture of conformational isomers comprising a first conformational isomer and a second conformational isomer; and
c) isolating the first conformational isomer, such as the most polar or the least polar of the conformational isomers of the compound of formula (I), or
d) forming a solution of the mixture of conformational isomers in a polar solvent, a non-polar solvent, such as chloroform, or a mixture of polar and non-polar solvents that produce a homogenous solution of the conformational isomers, allowing the second conformational isomer to convert to the first conformational isomer and isolating the total amount of the first conformational isomer, and
optionally d) hydrolyzing the isolated conformational isomer under basic conditions,
wherein when R⁶ comprises a precursor of an electrophilic group, the process further optionally comprises a step of converting the precursor of the electrophilic group to the electrophilic group,
wherein when R⁶ comprises a precursor or protected form of a nucleophilic group, the process further optionally comprises a step of converting the precursor or protected form of the nucleophilic group to the nucleophilic group,
wherein when R⁶ comprises an electrophilic group, the process further optionally comprises a step of converting the electrophilic group to a nucleophilic group, and
wherein when R⁶ comprises a nucleophilic group, the process further optionally comprises a step of converting the nucleophilic group to an electrophilic group.

2. The isolated conformational isomer according to claim 1, wherein Q² is 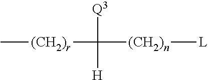
wherein n, r, L, Q³ and L are as defined above.

3. The isolated conformational isomer of claim 2, wherein Q³ is —(CHR²)_{w}CO₂R³, wherein w, R² and R³ are as defined above.

4. The isolated conformational isomer of claim 2, wherein Q¹ is —(CHR²)ₚCO₂R³, wherein p, R² and R³ are as defined above.

5. The isolated conformational isomer according to claim 1, wherein Q² is 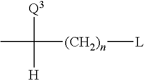
wherein n, L and Q³ are as defined above.

6. The isolated conformational isomer of claim 5, wherein Q³ is —CO₂R³, wherein R³ is as defined above.

7. The isolated conformational isomer of claim 5, wherein Q¹ is —(CHR²)ₚCO₂R³, wherein p, R² and R³ are as defined above.

8. The isolated conformational isomer of claim 5, wherein Q¹ is —CHR²CO₂R³, wherein R² and R³ are as defined above.

9. The isolated conformational isomer of claim 5, wherein Q¹ is —CH₂CO₂R³, wherein R² and R³ are as defined above.

10. The isolated conformational isomer according to claim 1, wherein Q² is 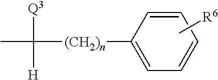
wherein n, Q³ and R⁶ are as defined above.

11. The isolated conformational isomer of claim 10, wherein Q³ is —CO₂R³, wherein R³ is as defined above.

12. The isolated conformational isomer of claim 10, wherein Q¹ is —(CHR²)ₚCO₂R³, wherein p, R² and R³ are as defined above.

13. The isolated conformational isomer of claim 10, wherein Q¹ is —CHR²CO₂R³, wherein R² and R³ are as defined above.

14. The isolated conformational isomer of claim 10, wherein Q¹ is —CH₂CO₂R³, wherein R² and R³ are as defined above.

15. The isolated conformational isomer according to claim 1, wherein the isolated conformational isomer is of the formula:

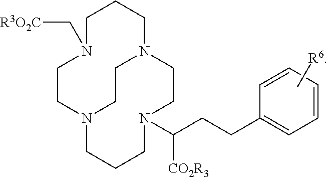

16. The isolated conformational isomer according to claim 1, wherein the isolated conformational isomer is of the formula:

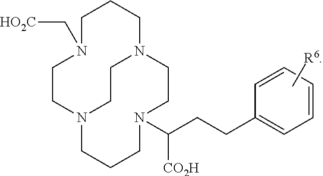

17. The isolated conformational isomer according to claim 1, wherein R⁶ is NO₂, NH₂, isothiocyanato, semicarbazido, thiosemicarbazido, maleimido, bromoacetamido or carboxylic acid.

18. A complex comprising the isolated conformational isomer defined in claim 1 or a pharmaceutically acceptable salt thereof, and an ion of a stable or radioactive form of Cu.

19. The complex according to claim 18, wherein the ion is selected from a group consisting of ⁶⁰Cu²⁺, ⁶²Cu²⁺, ⁶⁴Cu²⁺ and ⁶⁷Cu²⁺.

20. A conjugate comprising the complex of claim 18 covalently attached to a biological carrier.

21. A conjugate comprising the complex of claim 18 and a biological carrier attached to a nanoparticle.

22. The conjugate according to claim 20, wherein the biological carrier is a protein, antibody, antibody fragment, hormone, peptide, growth factor, antigen or hapten.

23. A process for chelating the isolated conformational isomer defined claim 1 with an ion of a stable or radioactive form of Cu, comprising contacting the isolated conformational isomer with the ion and allowing a complex between the isolated conformational isomer and the ion to form.

24. A method of isolating a conformational isomer of a bifunctional chelating agent of formula (I): 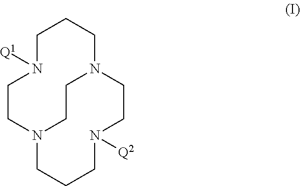
wherein:
Q¹ is —(CHR²)ₚCO₂R³ or —(CHR²)ₚPO₃R⁴R⁵;
Q² is 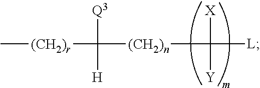
Q³ is —(CHR²)_{w}CO₂R³ or —(CHR²)_{w}PO₃R⁴R⁵;
each R² is independently hydrogen; C₁-C₄ alkyl or (C₁-C₂alkyl)phenyl;
each R³ is independently H, benzyl, C₁-C₄ alkyl, or a protecting group;
R⁴ and R⁵ are independently H, C₁-C₆ alkyl, (C₁-C₂ alkyl)phenyl or a protecting group;
X and Y are each independently hydrogen or may be taken with an adjacent X and Y to form an additional carbon-carbon bond;
m is an integer from 0 to 5 inclusive;
n is an integer from 1 to 5 inclusive;
p is 1 or 2;
r is 0 or 1;
w is 0 or 1;
L is a linker/spacer group covalently bonded to a carbon atom and replaces one hydrogen atom of said carbon atom, said linker/spacer group being represented by the formula: 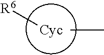
wherein:
R⁶ is H or an electrophilic, nucleophilic or electron-rich moiety which allows for covalent attachment to a biological carrier, or synthetic linker which can be attached to a biological carrier, a protected form thereof or a precursor thereof; and
Cyc represents a linear, branched or cyclic aliphatic moiety, aromatic moiety, aliphatic heterocyclic moiety, or aromatic heterocyclic moiety, each of said moieties optionally substituted with one or more groups which do not interfere with binding to a biological carrier;
or a pharmaceutically acceptable salt thereof, the method comprising:
a) reacting a compound of formula: 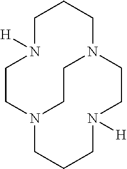
with a compound of formula Q²-LG, wherein Q² is as defined above and LG is a leaving group, to form a mixture of conformational isomers of a mono-alkylated derivative of formula: 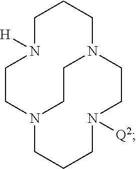
b) reacting the mixture of conformational isomers of the monoalkylated derivative with a compound of formula Q¹-LG, wherein Q¹ is as defined above and LG is a leaving group, to form a mixture of conformational isomers of the compound of formula (I), the mixture of conformational isomers comprising a first conformational isomer and a second conformational isomer; and
c) isolating the first conformational isomer, such as the most polar or the least polar of the conformational isomers of the compound of formula (I), or
d) forming a solution of the mixture of conformational isomers in a polar solvent, a non-polar solvent, such as chloroform, or a mixture of polar and non-polar solvents that produce a homogenous solution of the conformational isomers, allowing the second conformational isomer to convert to the first conformational isomer and isolating the total amount of the first conformational isomer, and
optionally e) hydrolyzing the isolated conformational isomer under basic conditions,
wherein when R⁶ comprises a precursor of an electrophilic group, the process further optionally comprises a step of converting the precursor of the electrophilic group to the electrophilic group,
wherein when R⁶ comprises a precursor or protected form of a nucleophilic group, the process further optionally comprises a step of converting the precursor or protected form of the nucleophilic group to the nucleophilic group,
wherein when R⁶ comprises an electrophilic group, the process further optionally comprises a step of converting the electrophilic group to a nucleophilic group, and
wherein when R⁶ comprises a nucleophilic group, the process further optionally comprises a step of converting the nucleophilic group to an electrophilic group.

25. The method according to claim 24, wherein Q² is 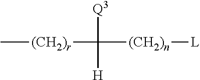
wherein n, r, L, Q³ and L are as defined above.

26. The method of claim 25, wherein Q³ is —(CHR²)_{w}CO₂R³, wherein w, R² and R³ are as defined above.

27. The method of claim 25, wherein Q¹ is —(CHR²)ₚCO₂R³, wherein p, R² and R³ are as defined above.

28. The method according to claim 24, wherein Q² is 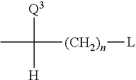
wherein n, L and Q³ are as defined above.

29. The method of claim 28, wherein Q³ is —CO₂R³, wherein R³ is as defined above.

30. The method of claim 28, wherein Q¹ is —(CHR²)ₚCO₂R³, wherein p, R² and R³ are as defined above.

31. The method of claim 28, wherein Q¹ is —CHR²CO₂R³, wherein R² and R³ are as defined above.

32. The method of claim 28, wherein Q¹ is —CH₂CO₂R³, wherein R² and R³ are as defined above.

33. The method according to claim 24, wherein Q² is 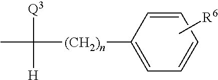
wherein n, Q³ and R⁶ are as defined above.

34. The method of claim 33, wherein Q³ is —CO₂R³, wherein R³ is as defined above.

35. The method of claim 33, wherein Q¹ is —(CHR²)ₚCO₂R³, wherein p, R² and R³ are as defined above.

36. The method of claim 33, wherein Q¹ is —CHR²CO₂R³, wherein R² and R³ are as defined above.

37. The method of claim 33, wherein Q¹ is —CH₂CO₂R³, wherein R² and R³ are as defined above.

38. The method according to claim 24, wherein the isolated conformational isomer is of the formula:

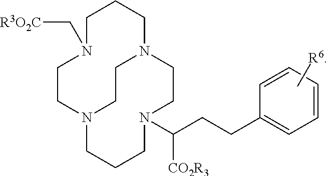

39. The method according to claim 24, wherein the isolated conformational isomer is of the formula:

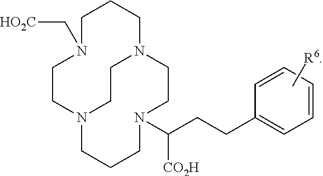

40. The method according to claim 24, wherein R⁶ is NO₂, NH₂, isothiocyanato, semicarbazido, thiosemicarbazido, maleimido, bromoacetamido or carboxylic acid.

41. A method of converting a first conformational isomer of a bifunctional chelating agent of formula (I) into a second conformational isomer of formula (I): 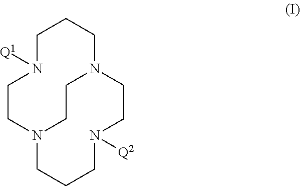
wherein:
Q¹ is —(CHR²)ₚCO₂R³ or —(CHR²)ₚPO₃R⁴R⁵;
Q² is 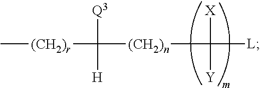
Q³ is —(CHR²)_{w}CO₂R³ or —(CHR²)_{w}PO₃R⁴R⁵;
each R² is independently hydrogen; C₁-C₄ alkyl or (C₁-C₂alkyl)phenyl;
each R³ is independently H, benzyl, C₁-C₄ alkyl, or a protecting group;
R⁴ and R⁵ are independently H, C₁-C₆ alkyl, (C₁-C₂ alkyl)phenyl or a protecting group;
X and Y are each independently hydrogen or may be taken with an adjacent X and Y to form an additional carbon-carbon bond;
m is an integer from 0 to 5 inclusive;
n is an integer from 1 to 5 inclusive;
p is 1 or 2;
r is 0 or 1;
w is 0 or 1;
L is a linker/spacer group covalently bonded to a carbon atom and replaces one hydrogen atom of said carbon atom, said linker/spacer group being represented by the formula: 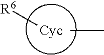
wherein:
R⁶ is H or an electrophilic, nucleophilic or electron-rich moiety which allows for covalent attachment to a biological carrier, or synthetic linker which can be attached to a biological carrier, a protected form thereof or a precursor thereof; and
Cyc represents a linear, branched or cyclic aliphatic moiety, aromatic moiety, aliphatic heterocyclic moiety, or aromatic heterocyclic moiety, each of said moieties optionally substituted with one or more groups which do not interfere with binding to a biological carrier;
or a pharmaceutically acceptable salt thereof, the method comprising:
forming a solution of the first conformational isomer and the second conformational isomer in a polar solvent, a non-polar solvent, such as chloroform, or a mixture of polar and non-polar solvents that produce a homogenous solution of the conformational isomers;
allowing the second conformational isomer to convert to the first conformational isomer,
isolating the total amount of the first conformational isomer, and
optionally e) hydrolyzing the isolated conformational isomer under basic conditions,
wherein when R⁶ comprises a precursor of an electrophilic group, the process further optionally comprises a step of converting the precursor of the electrophilic group to the electrophilic group,
wherein when R⁶ comprises a precursor or protected form of a nucleophilic group, the process further optionally comprises a step of converting the precursor or protected form of the nucleophilic group to the nucleophilic group,
wherein when R⁶ comprises an electrophilic group, the process further optionally comprises a step of converting the electrophilic group to a nucleophilic group, and
wherein when R⁶ comprises a nucleophilic group, the process further optionally comprises a step of converting the nucleophilic group to an electrophilic group.

42. The method according to claim 41, wherein the first conformation isomer is relatively more polar than the second conformational isomer.
